(19) United States
(12) Patent Application Publication
(10) Pub. No.: US 2024/0269295 A1
(43) Pub. Date: Aug. 15, 2024

(54) C-MET TARGETED APTAMER DRUG CONJUGATE AND METHOD FOR TREATING TUMOR

(71) Applicant: Hangzhou Institute of Medicine, Chinese Academy of Sciences, Hangzhou City (CN)

(72) Inventors: Weihong TAN, Hangzhou City (CN); Xiangsheng LIU, Hangzhou City (CN); Wencan WU, Hangzhou City (CN); Jiaxuan HE, Hangzhou City (CN); Yuan LIU, Hangzhou City (CN); Zhaoqi PAN, Hangzhou City (CN); Ting FU, Hangzhou City (CN); Sitao XIE, Hangzhou City (CN)

(21) Appl. No.: 18/431,688

(22) Filed: Feb. 2, 2024

(30) Foreign Application Priority Data
Feb. 3, 2023 (CN) 202310129381.2

(51) Int. Cl. A61K 47/54; A61P 35/00

(52) U.S. Cl. CPC A61K 47/549; A61P 35/00

(57) ABSTRACT

The present application relates to the field of biotechnology, and in particular to a c-Met targeted aptamer drug conjugate. The drug conjugate c-Met-ApDC of the present application can achieve a cytotoxic IC₅₀ of 100 nM in a cell with high expression of c-Met and a tumor model with high expression of c-Met, effectively inhibit a tumor in an animal model, achieve a tumor inhibition rate of 100% for a triple negative breast cancer PDX model, and gain complete regression of a transplanted tumor. The inhibition rate of the drug conjugate administrated subcutaneously to choroidal melanoma as a subcutaneous tumor by tail vein is up to 98.13%, and the inhibition rate of the drug conjugate injected intravitreally into intraocular choroidal melanoma in situ is 100%, which realizes complete regression of the transplanted tumor.

### CROSS-REFERENCE TO RELATED APPLICATIONS

The present application claims the priority of the prior Chinese application No. 202310129381.2 filed on Feb. 3, 2023, the content of which, including but not limited to the Specification, Claims, Abstract, and Drawings of Specification, is incorporated as a part of the present application in its entirety.

### REFERENCE TO AN ELECTRONIC SEQUENCE LISTING

The content of the electronic sequence listing (Sequence Listing.xml; Size: 2,955 bytes; and Date of Creation: Feb. 1, 2024) is herein incorporated by reference in its entirety.

### BACKGROUND OF THE INVENTION

### Field of the Invention

The present application relates to the field of biotechnology, and in particular to a c-Met targeted aptamer drug conjugate.

### Description of the Related Art

An aptamer is an antibody-like oligonucleotide sequence that can specifically bind to a target protein. The aptamer can be chemically synthesized, is easier to modify and more stable than an antibody, and has a smaller size and body immune response, so it has wide application in diagnosing and treating tumors. Conjugating a drug molecule with the aptamer can improve the water solubility of the drug molecule and enrichment of the same in a tumor cell, reduce the toxic and side effects of the drug molecule on a normal cell, and realize targeted delivery of the drug molecule.

C-Met is a protein (UniProt ID: P08581) encoded by a c-Met proto-oncogene called a hepatocyte growth factor receptor. It has tyrosine kinase activity, is related to many oncogene products and regulatory proteins, regulates cell information transduction and cytoskeleton rearrangement, and is an essential factor in cell proliferation, differentiation, and movement. The c-Met is believed to be closely related to the occurrence and metastasis of various cancers. Studies have shown that patients with lung cancer, gastric cancer, liver cancer, breast cancer, skin cancer, colorectal cancer, and the like tumors all have over-expression and gene amplification of the c-Met during the occurrence and metastasis of their tumors. Therefore, the c-Met is a compelling target for many tumors, and many c-Met-targeted drugs have entered the clinical stage. The c-Met targeted drugs under development are expected to become broad-spectrum targeted drugs for treating lung, gastric, liver, and malignant tumors.

A known aptamer SL1 is obtained by screening with systematic evolution of ligands by exponential enrichment (SELEX). It can specifically bind to the c-Met protein on the surface of a cell to recognize the target cell with high affinity, thereby internalizing into the cell. The aptamer SL1 has become a classical recognition probe for tumor cell molecules and has been widely applied in tumor diagnosis and treatment, but its effect is limited.

In the prior art, an aptamer Ap3 is already conjugated with a tubulin inhibitor to prepare drugs for treating lung cancer, colon cancer, and pancreatic cancer. Still, the drug effects are poor in animal models, and the pharmaceutical-specific impact in other cancers is unknown. Improving the existing traditional technology and increasing the therapeutic effect is necessary.

### BRIEF SUMMARY OF THE INVENTION

Because of the shortcomings of the existing traditional technology mentioned above, to solve the technical problem of a poor pharmaceutical effect of the drug for preventing or treating malignant tumors in the current conventional technology, an objective of the present application is to provide a c-Met targeted aptamer drug conjugate, which conjugates a c-Met targeted aptamer with a cytotoxic drug commonly used in targeted drugs, namely auristatin tubulin inhibitor or a derivative thereof, to realize the targeted delivery of the auristatin tubulin inhibitor or the derivative thereof and realize the targeted treatment of malignant tumors, and can be used for solving the problems in the prior art.

To achieve the objectives above and other related purposes, the first aspect of the present application provides a drug conjugate, which includes a conjugated aptamer SL1 and a tubulin inhibitor.

In any embodiment of the present application, a sequence of the aptamer SL1 is as shown in SEQ ID NO:1. Specifically, it is

(SEQ ID NO: 1)
ATCAGGCTGGATGGTAGCTCGGTCGGGGTGGGTGGGTTGGCAAGTCTGA
T.

In any embodiment of the present application, the tubulin inhibitor is selected from an auristatin tubulin inhibitor, and preferably, the auristatin tubulin inhibitor is selected from monomethyl auristatin E, monomethyl auristatin F, auristatin-0101 or derivatives thereof.

In any embodiment of the present application, the conjugating mode is covalent conjugating.

In any embodiment of the present application, the tubulin inhibitor further includes a first linking group for linking the aptamer, and the aptamer includes a second linking group for connecting the tubulin inhibitor; preferably, the first linking group is selected from mercapto or amino, and the second linking group is assigned from carboxyl or azido. Here, the second linking group is also a group that modifies the aptamer, and the aptamer is conjugated with a targeted drug after modification. In some embodiments, the amino group is maleimide.

In any embodiment of the present application, the aptamer is conjugated with a tubulin inhibitor after 3-terminal mercapto modification. Through our experiments, it has been found that the 3-terminal mercapto modification significantly improves the stability of the aptamer compared with the 5-terminal mercapto modification. More preferably, the terminal modification of the aptamer is selected from mercapto modification or amino modification.

In any embodiment of the present application, the chemical formula of the drug conjugate is selected from

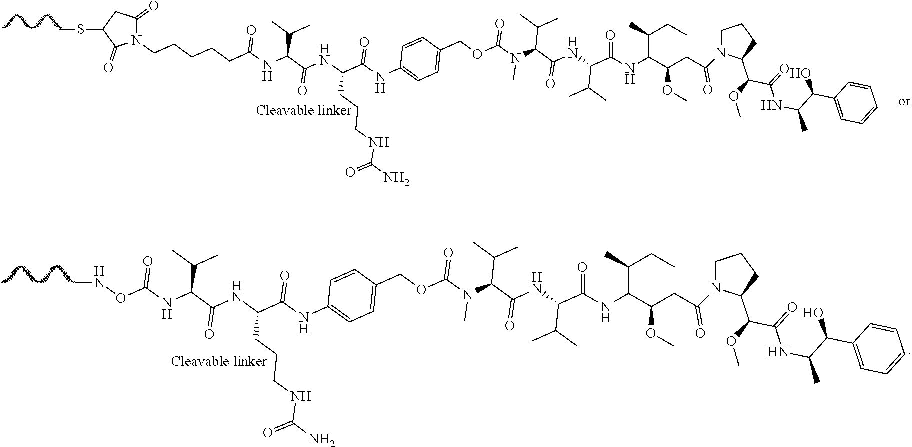

wherein [symbol] represents the aptamer.

A second aspect of the present application provides a method for preparing a drug conjugate, which includes the following steps:
1) mixing and stirring an aptamer solution and a tubulin inhibitor solution for a conjugating reaction to obtain a crude product and
2) purifying the crude product of the step 1) to obtain the drug conjugate.

In any embodiment of the present application, in step 1), a solvent for the aptamer and a solvent for the tubulin inhibitor are each selected from a combination of one or more nuclease-free water, a phosphate buffer, a triethylammonium acetate buffer, and acetonitrile.

In any embodiment of the present application, in step 1), the reaction time is 3-36 h.

In any embodiment of the present application, in step 1), the reaction temperature is 4-40° C.

In any embodiment of the present application, in step 1), the tubulin inhibitor has 1-5 equivalents; preferably, the tubulin inhibitor has three equivalents.

In any embodiment of the present application, in step 1), the solvent for the aptamer or the solvent for the tubulin inhibitor is 0.1-50 mL.

In any embodiment of the present application, in step 2), the purification is selected from a combination of one or more high-performance liquid chromatography, size exclusion chromatography, and gel electrophoresis.

A third aspect of the present application provides the drug conjugate of the first aspect or the preparation method of the second aspect in preparing a product for preventing or treating a malignant tumor. In any embodiment of the present application, the malignant tumor is selected from a malignant tumor with a high expression of c-Met. In any embodiment of the present application, the malignant tumor with increased expression of c-Met is selected from triple-negative breast cancer, colon cancer, lung cancer, gastric cancer, or choroidal melanoma; and preferably, the malignant tumor with high expression of c-Met is selected from triple-negative breast cancer and choroidal melanoma.

A fourth aspect of the present invention provides a method for treating a tumor, including applying a drug conjugate to a subject so that the conjugate binds to a c-Met protein in the body of the subject in a targeted manner, thereby bringing the drug into a tumor cell to inhibit the proliferation of the tumor cell, wherein the subject includes human and a mammal. The drug conjugate consists of an aptamer SL1 and a tubulin inhibitor. Here, the mode of application can be intravenous injection. In some embodiments, a sequence of the aptamer SL1 is shown in SEQ ID NO: 1. In some embodiments; the tubulin inhibitor is selected from an auristatin tubulin inhibitor.

In some embodiments, it is characterized in that the conjugating is selected from covalent conjugating. In some embodiments, the tubulin inhibitor further includes a first linking group for linking an aptamer, and the aptamer includes a second linking group for connecting the tubulin inhibitor; the first linking group is selected from mercapto or amino, and the second linking group is assigned from carboxyl or azido, so that the first group is linked with the second group, thereby covalently conjugating the aptamer SL1 with the tubulin inhibitor. In some embodiments, the aptamer is conjugated with the tubulin inhibitor after stability modification; the stability modification is a 3-terminal modification, and the terminal modification is selected from mercapto modification or amino modification. In some embodiments, the chemical formula of the drug conjugate is selected from

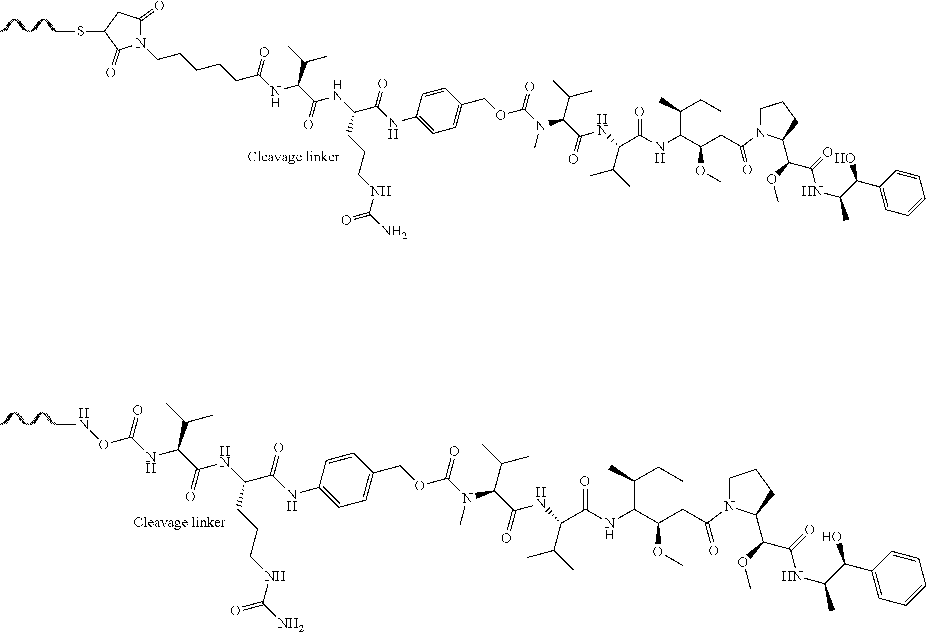 [SMILES: CC[C@H](C)C(NC(=O)[C@@H](NC(=O)[C@H](C(C)C)N(C)C(=O)OCc1ccc(NC(=O)[C@H](CCCNC(N)=O)NC(=O)[C@@H](NC(=O)CCCCCN2C(=O)CC(SC)C2=O)C(C)C)cc1)C(C)C)[C@@H](CC(=O)N1CCC[C@H]1[C@H](OC)C(=O)N[C@H](C)[C@@H](O)c1ccccc1)OC ; CC[C@H](C)C(NC(=O)[C@@H](NC(=O)[C@H](C(C)C)N(C)C(=O)OCc1ccc(NC(=O)[C@H](CCCNC(N)=O)NC(=O)[C@@H](NC(=O)ONC)C(C)C)cc1)C(C)C)[C@@H](CC(=O)N1CCC[C@H]1[C@H](OC)C(=O)N[C@H](C)[C@@H](O)c1ccccc1)OC]

In some embodiments, the malignant tumor is selected from a malignant tumor with a high expression of c-Met. In some embodiments, the malignant tumor with increased expression of c-Met is selected from triple-negative breast cancer, colon cancer, lung cancer, gastric cancer, or choroidal melanoma. In some embodiments, the malignant tumor with high expression of c-Met is triple-negative breast cancer or colon cancer. In some embodiments, intravenous and intravitreal administration is adopted as the mode of administration when choroidal melanoma is treated.

Compared with the prior art, the present application has the following beneficial effects.
1. The drug conjugate c-Met-ApDC of the present application can achieve a cytotoxic IC50 of 100 nM in a cell with high expression of c-Met and a tumor model with high expression of c-Met, effectively inhibit a tumor in an animal model, achieve a tumor inhibition rate of 100% for a tumor in a triple-negative breast cancer PDX model and choroidal melanoma, and achieve complete healing.
2. Compared with a standard chemotherapy Docetaxel group, the drug conjugate c-Met-ApDC of the present application has a significant inhibitory effect and achieves an inhibitory rate of 90.6%. Meanwhile, aiming at the fact that Docetaxel chemotherapy cannot inhibit the tumor, the tumor can be significantly reduced after administration of the drug conjugate. This shows that the proliferation of cells with high c-Met expression and tumors with high expression is effectively inhibited.
3. In the treatment of in situ choroidal melanoma with high expression of c-Met, the drug conjugate c-Met-ApDC of the present application is administered through intravitreal administration, which achieves 100% inhibition of the in situ choroidal melanoma.

### BRIEF DESCRIPTION OF THE DRAWINGS

FIG. 1 shows the characterization of a c-Met targeted aptamer drug conjugate by mass spectrometry.

FIG. 2 shows a cell proliferation inhibition experiment of a c-Met targeted aptamer drug conjugate in skov3 cells.

FIG. 3 shows a cell proliferation inhibition experiment of a c-Met targeted aptamer drug conjugate in A549 cells.

FIG. 4 shows a cell proliferation inhibition experiment of a c-Met targeted aptamer drug conjugate in MKN45 cells.

FIG. 5 shows a cell proliferation inhibition experiment of a c-Met targeted aptamer drug conjugate in MUM2b cells.

FIG. 6 shows the tumor volume against triple-negative breast cancer PDX model tumors in vivo.

FIG. 7 shows a relative tumor volume in the experiment of a c-Met targeted aptamer drug conjugate against triple-negative breast cancer PDX model tumors in vivo.

FIG. 8 shows the body weights of mice in the experiment of a c-Met targeted aptamer drug conjugate against triple-negative breast cancer PDX model tumors in vivo.

FIG. 9 shows the relative body weights of mice in the experiment of a c-Met targeted aptamer drug conjugate against triple-negative breast cancer PDX model tumors in vivo.

FIG. 10 shows the tumor immunohistochemistry after treatment of triple-negative breast cancer PDX model tumors with the c-Met targeted aptamer drug conjugate.

FIG. 11 shows the toxicity on bone marrow and liver after treatment of triple-negative breast cancer PDX model tumors with the c-Met targeted aptamer drug conjugate.

FIG. 12 shows a tumor volume in an experiment of the c-Met targeted aptamer drug conjugate against large triple-negative breast cancer PDX model tumors in vivo.

FIG. 13 shows a relative tumor volume in the experiment of a c-Met targeted aptamer drug conjugate against large triple-negative breast cancer PDX model tumors in vivo.

FIG. 14 shows a tumor volume in an experiment of the c-Met targeted aptamer drug conjugate against large triple-negative breast cancer PDX model tumors in vivo after multiple times of administrations.

FIG. 15 shows the tumor volume of the triple-negative breast cancer PDX model under different administration dosages and frequencies of c-Met-ApDC.

FIG. 16 shows the body weights of triple-negative breast cancer PDX model mice under different administration dosages and frequencies of c-Met-ApDC.

FIG. 17 shows a tumor volume in an experiment of the c-Met targeted aptamer drug conjugate against an HT-29 tumor in vivo.

FIG. 18 shows the body weights of mice in the experiment of a c-Met targeted aptamer drug conjugate against a HT-29 tumor in vivo.

FIG. 19 shows a tumor volume in an experiment of the c-Met targeted aptamer drug conjugate against an A549 tumor in vivo.

FIG. 20 shows a tumor volume in an experiment of the c-Met targeted aptamer drug conjugate against an MKN45 tumor in vivo.

FIG. 21 shows a tumor volume in an experiment of a c-Met targeted aptamer drug conjugate against a choroidal melanoma tumor MUM2b in vivo via tail vein injection.

FIG. 22 shows a relative tumor volume in the experiment of a c-Met targeted aptamer drug conjugate against a choroidal melanoma tumor MUM2b in vivo via tail vein injection.

FIG. 23 shows the body weights of mice in an experiment of a c-Met targeted aptamer drug conjugate against a choroidal melanoma tumor MUM2b in vivo via tail vein injection.

FIG. 24 shows the relative body weights of mice in an experiment of a c-Met targeted aptamer drug conjugate against a choroidal melanoma tumor MUM2b in vivo via tail vein injection.

FIG. 25 shows a fluorescence signal in an experiment of a c-Met targeted aptamer drug conjugate against in situ choroidal melanoma cancer in vivo via intravitreal injection.

FIG. 26 shows the statistics of a fluorescence signal in the experiment of a c-Met targeted aptamer drug conjugate against in situ choroidal melanoma cancer in vivo via intravitreal injection.

FIG. 27 shows the statistics of a relative fluorescence signal in the experiment of a c-Met targeted aptamer drug conjugate against in situ choroidal melanoma cancer in vivo via intravitreal injection.

FIG. 28 shows the body weights of mice in an experiment of a c-Met targeted aptamer drug conjugate against an in situ choroidal melanoma tumor in vivo via intravitreal injection.

FIG. 29 shows the relative body weights of mice in an experiment of a c-Met targeted aptamer drug conjugate against an in situ choroidal melanoma tumor in vivo via intravitreal injection.

FIG. 30 shows the result of an experiment comparing a c-Met targeted aptamer drug conjugate and vcMMAE in inhibiting HT29 cell proliferation.

FIG. 31 shows a comparison of the inhibitory efficiency (on cell proliferation) of a c-Met targeted aptamer drug conjugate and vcMMAE on MNK45 tumor cells.

FIG. 32 shows a comparison of the inhibitory efficiency (on cell proliferation) of a c-Met targeted aptamer drug conjugate and vcMMAE on NCI-H1975 tumor cells.

FIG. 33 shows the result of an experiment comparing a c-Met targeted aptamer drug conjugate and vcMMAE in inhibition of MUM2b cell proliferation.

FIG. 34 is a graph of the result of an experiment comparing the stability of an aptamer conjugated with a drug at the 3-terminal and the aptamer conjugated with the drug at the 5-terminal.

FIG. 35 shows an experiment for comparing the binding affinity of different c-Met aptamer drug conjugates with a c-Met protein.

FIG. 36 is a graph of an experiment for comparing the cytotoxicity of different c-Met aptamer drug conjugates.

### DETAILED DESCRIPTION OF THE INVENTION

To make the invention objective, technical solutions, and beneficial effects of the present application clearer, the present application will be further illustrated in connection with examples hereafter. It should be understood that the examples are only used for explaining the present application rather than limiting the scope of the present application. Unless otherwise specified, the experiment methods used in the following examples are all conventional, and those skilled in the art can easily understand other advantages and efficacy of the present application from the disclosure in the description.

The inventor of the present application has found a c-Met targeted aptamer drug conjugate through a lot of exploration and research and completed the present application on this basis.

An aspect of the present application provides a drug conjugate, which includes a conjugated aptamer SL1 and a tubulin inhibitor.

In the drug conjugate provided by the present application, a sequence of the aptamer SL1 is as shown in SEQ ID NO:1. Specifically, it is

(SEQ ID NO: 1)
ATCAGGCTGGATGGTAGCTCGGTCGGGGTGGGTGGGTTGGCAAGTCTGA
T.

An aptamer SL1 is obtained by screening with cell-systematic evolution of ligands by exponential enrichment (cell-SELEX). It can specifically bind to the c-Met protein on the surface of a cell to recognize the target cell with high affinity, thereby internalizing into the cell. In some embodiments, all other aptamers targeting c-Met can be used in the present invention.

The tubulin inhibitor is selected from an auristatin tubulin inhibitor in the drug conjugate provided by the present application. Preferably, the auristatin tubulin inhibitor is selected from monomethyl auristatin E, monomethyl auristatin F, auristatin-0101, or derivatives thereof.

In some specific embodiments of the present application, the tubulin inhibitor is selected from vcMMAE or SuO-Val-Cit-PAB-MMAE. The vcMMAE (mc-vc-PAB-MMAE) is a part of an antibody-conjugate drug with anticancer activity. It is formed by linking MMAE (a tubulin inhibitor) to valine-citrulline (vc).

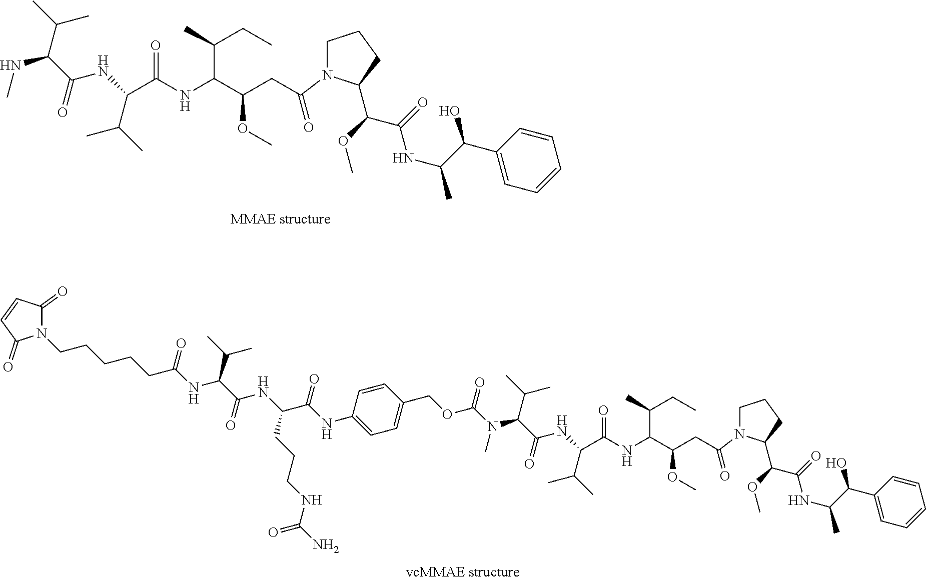 [SMILES: CC[C@H](C)C(NC(=O)[C@@H](NC(=O)[C@@H](NC)C(C)C)C(C)C)[C@@H](CC(=O)N1CCC[C@H]1[C@H](OC)C(=O)N[C@H](C)[C@@H](O)c1ccccc1)OC ; CC[C@H](C)C(NC(=O)[C@@H](NC(=O)[C@H](C(C)C)N(C)C(=O)OCc1ccc(NC(=O)[C@H](CCCNC(N)=O)NC(=O)[C@@H](NC(=O)CCCCCN2C(=O)C=CC2=O)C(C)C)cc1)C(C)C)[C@@H](CC(=O)N1CCC[C@H]1[C@H](OC)C(=O)N[C@H](C)[C@@H](O)c1ccccc1)OC]

SuO-Val-Cit-PAB-MMAE is part of an antibody-conjugate drug that links a dipeptide linker SuO-Val-Cit-PAB with an antimitotic agent (tubulin inhibitor) MMAE.

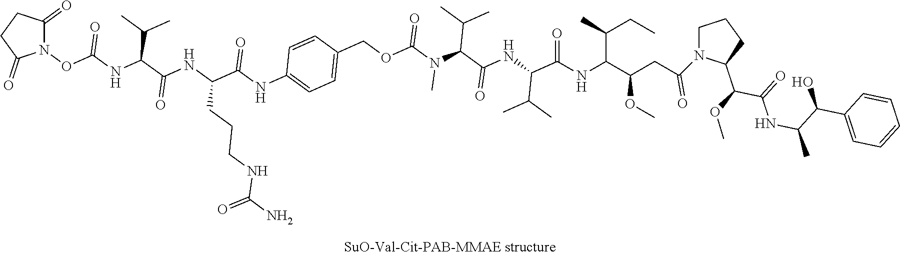 [SMILES: CC[C@H](C)C(NC(=O)[C@@H](NC(=O)[C@H](C(C)C)N(C)C(=O)OCc1ccc(NC(=O)[C@H](CCCNC(N)=O)NC(=O)[C@@H](NC(=O)ON2C(=O)CCC2=O)C(C)C)cc1)C(C)C)[C@@H](CC(=O)N1CCC[C@H]1[C@H](OC)C(=O)N[C@H](C)[C@@H](O)c1ccccc1)OC]

A tubulin inhibitor acts on a microtubule system and is one class of effective anti-tumor drugs. Tubulin has the kinetic characteristics of polymerization and depolymerization and plays a vital role in cell morphology, cell division, signal transduction, substance transport, and processes. The tubulin is polymerized into a spindle in the prophase of cell division, and the spindle pulls chromosomes to move towards two poles in mitosis and enter two daughter cells to complete cell proliferation. The tubulin inhibitor plays an essential role in a tumor drug, which can inhibit cell division, thereby inhibiting tumor cell division and ultimately inhibiting tumor cell proliferation.

In the drug conjugate provided by the present application, the conjugation is selected from covalent conjugation.

In the drug conjugate provided by the present application, the aptamer further includes a first linking group for linking a tubulin inhibitor, and the tubulin inhibitor further comprises a second linking group for linking the aptamer. In a specific embodiment of the present application, the first linking group is selected from mercapto or amino, and the second linking group is assigned from carboxyl or azido.

In some embodiments, the first linking group can be linked with the aptamer to play a modification role. The second group can be connected with an active ingredient and then linked with the first group, so the aptamer is coupled with the tubulin inhibitor.

The aptamer is conjugated with a tubulin inhibitor after stability modification in the drug conjugate provided by the present application, The stability modification is terminal, selected from mercapto modification or amino modification.

In the drug conjugate provided by the present application, the chemical formula of the drug conjugate is selected from

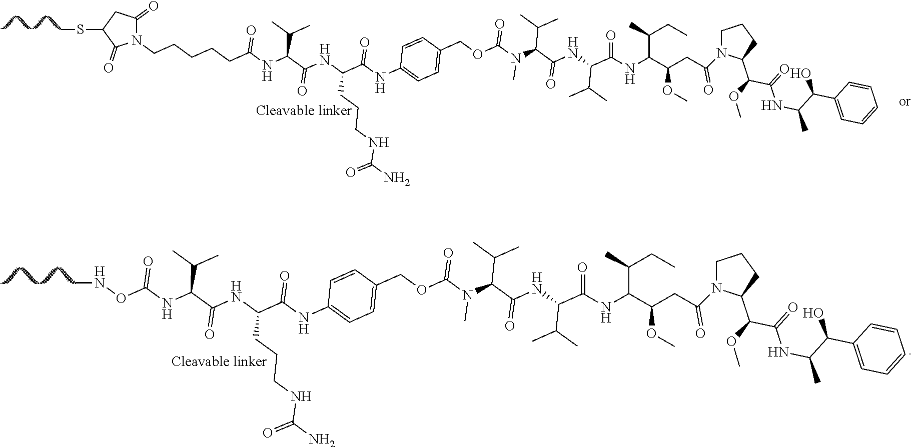 [SMILES: CC[C@H](C)C(NC(=O)[C@@H](NC(=O)[C@H](C(C)C)N(C)C(=O)OCc1ccc(NC(=O)[C@H](CCCNC(N)=O)NC(=O)[C@@H](NC(=O)CCCCCN2C(=O)CC(SC)C2=O)C(C)C)cc1)C(C)C)[C@@H](CC(=O)N1CCC[C@H]1[C@H](OC)C(=O)N[C@H](C)[C@@H](O)c1ccccc1)OC ; CC[C@H](C)C(NC(=O)[C@@H](NC(=O)[C@H](C(C)C)N(C)C(=O)OCc1ccc(NC(=O)[C@H](CCCNC(N)=O)NC(=O)[C@@H](NC(=O)ONC)C(C)C)cc1)C(C)C)[C@@H](CC(=O)N1CCC[C@H]1[C@H](OC)C(=O)N[C@H](C)[C@@H](O)c1ccccc1)OC]

Another aspect of the present application provides a method for preparing the preceding drug conjugate, which includes the following steps:
1) mixing and stirring an aptamer solution and a tubulin inhibitor solution for a conjugating reaction to obtain a crude product and
2) purifying the crude product of the step 1) to obtain the drug conjugate.

In the preparation method provided by the present application, step 1) refers to mixing and stirring an aptamer solution and a tubulin inhibitor solution for a conjugating reaction to obtain a crude product. In step 1), a solvent for the aptamer and a solvent for the tubulin inhibitor are each selected from a combination of one or more nuclease-free water, a phosphate buffer, a triethylammonium acetate buffer, and acetonitrile.

In a specific embodiment of the present application, the solvent for the aptamer is nuclease-free water, and the solvent for the tubulin inhibitor is a mixed solvent of acetonitrile and nuclease-free water. The tubulin inhibitor solution is 2-6 equivalents. In a preferred embodiment of the present application, the tubulin inhibitor solution is three equivalents, and the aptamer solution is one equivalent.

In the preparation method provided by the present application, in step 1), the conjugating reaction is selected from a mercapto-maleimide, azido-alkyne, or amino-carboxyl conjugating reaction.

In some embodiments, the conjugating chemical reaction formula of a mercapto-modified c-Met targeted aptamer and an auristatin tubulin inhibitor (vcMMAE) is as follows:

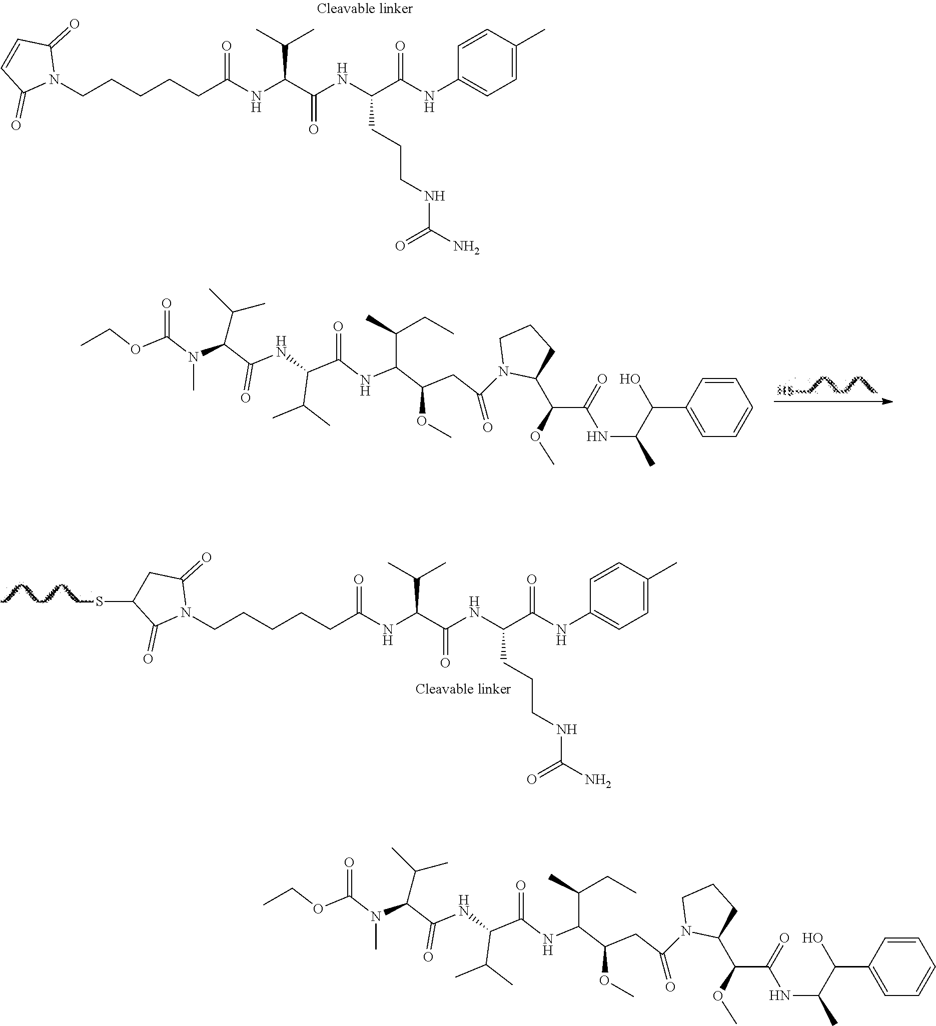

wherein the mercapto-bearing ribbon is the aptamer.

In some embodiments, the conjugating chemical reaction formula of an amino-modified c-Met targeted aptamer and an auristatin tubulin inhibitor (SuO-Val-Cit-PAB-MMAE) is as follows:

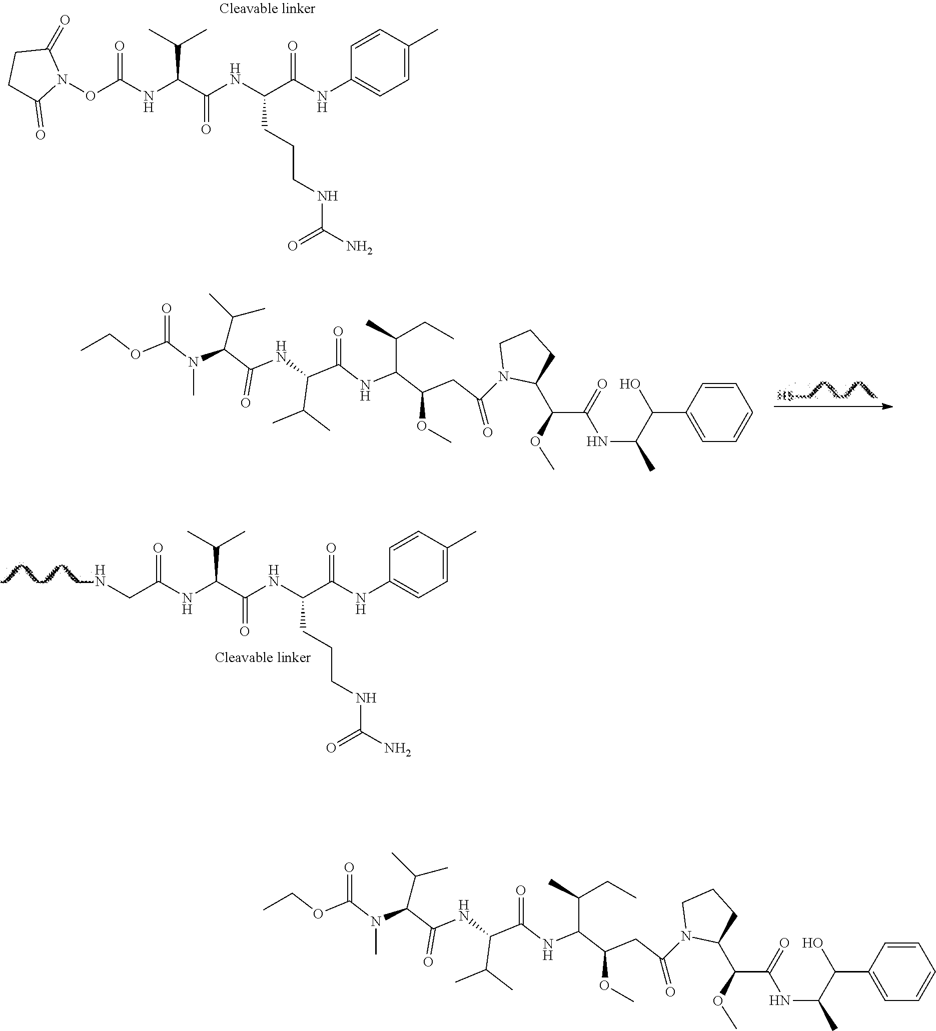

wherein the amino-bearing ribbon is the aptamer.

In the preparation method provided by the present application, in step 1), the reaction time is 3-36 h. In a preferred embodiment of the present application, the reaction time is 16 h.

In the preparation method provided by the present application, in step 1), a reaction temperature is 4-40° C. In a preferred embodiment of the present application, the reaction temperature is 37° C.

In step 1), the tubulin inhibitor is 1-5 equivalents in the preparation method provided by the present application. The tubulin inhibitor has three equivalents in a preferred embodiment of the present application.

In the preparation method provided by the present application, step 1), the solvent for the aptamer or the solvent for the tubulin inhibitor is 0.1-50 mL.

In the preparation method provided by the present application, in step 2), the purification is selected from a combination of one or more high-performance liquid chromatography, size exclusion chromatography, and gel electrophoresis.

A further aspect of the present application provides the use of the preceding drug conjugate or the preceding preparation method in preparing a product for preventing or treating malignant tumors or a method for treating cancer. In some embodiments, the malignant tumor is selected from a malignant tumor with a high expression of c-Met. The malignant tumor with increased expression of c-Met is selected from triple-negative breast cancer, colon cancer, lung cancer, or gastric cancer; preferably, the malignant tumor with high c-Met expression is selected from triple-negative breast cancer.

In some embodiments of the present application, cell lines and animal models with high c-Met expression are used to simulate malignant tumors in vitro and in vivo environments with high c-Met expression, respectively. The cell line with high c-Met expression is selected from SKOV-3, A549, or MKN45. Specifically, SKOV-3 is an ovarian cancer cell, A549 is a non-small cell lung cancer cell, MKN45 is a gastric cancer cell, and MUM2b is a human invasive choroidal melanoma cell.

In some embodiments, the animal model with high expression of c-Met is selected from a tumor-bearing mouse model with high expression of c-Met, such as a human colon cancer cell HT-29, a non-small cell lung cancer cell A549, a human gastric cancer cell MKN45 and a human choroidal melanoma cell MUM2b; or a triple negative breast cancer PDX model. The drug conjugate c-Met-ApDC of the present application can achieve a cytotoxic IC50 of 100 nM in a cell with high expression of c-Met and a tumor model with high expression of c-Met, effectively inhibit a tumor in an animal model, achieve a tumor inhibition rate of 100% for a tumor in a triple negative breast cancer PDX model, and gain complete healing.

### SPECIFIC EXAMPLES

The following examples will further illustrate the present application, but the scope of the present application is not limited thereby.

### Experimental Materials and Reagents

BALB/c female nude mice are used as a mouse model, and a tumor mass of triple negative breast cancer PDX is transplanted with a trocar.

Medium formula: RPMI-1640+10% fetal bovine serum (FBS)+1% penicillin-streptomycin solution (.PS) RPMI-1640: available from Meilunbio, article number MA0215-2FBS: available from Nanjing BioChannel Biotechnology Co., Ltd., article number BC-SE-FBSO74Ps: available from Meilunbio, article number MA0233.

Drug-containing medium: a drug solution with a concentration of 50 uM is diluted with a medium to prepare a drug-containing medium with a concentration of 1,000 nM, and then the drug-containing medium is diluted in gradients to obtain drug-containing media with concentrations of 500 nM, 250 nM, 125 nM, 62.5 nM, 31.25 nM, and 15.625 nM.

The purification by reversed-phase preparative column, desalination, seed plating, tail vein administration, flow cytometry experiments, and tumor transplantation in cells involved in the following examples are all conventional methods in the art, which can be determined by those skilled in the art in combination with the common knowledge in the art and the contents recorded in the present invention.

### Example 1: Conjugating of c-Met Targeted Aptamer with Auristatin Tubulin Inhibitor (vcMMAE)

### The Synthesis Route of a Mercapto-Maleimide Chemical Reaction was as Follows (the Mercapto-Bearing Ribbon was an Aptamer Modified by a Mercapto Group):

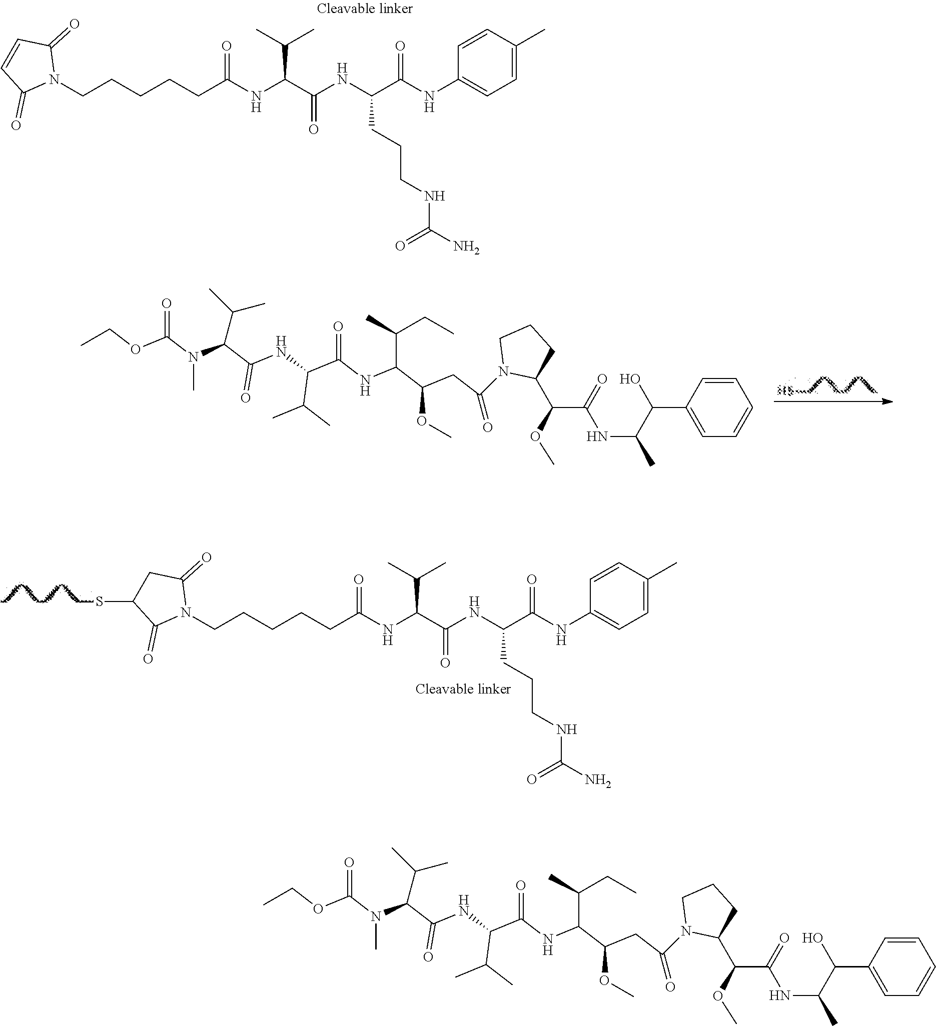

An aptamer sequence is shown in SEQ ID NO:1. A nucleic acid sequence conjugated with a drug at the 3-terminal was adopted.

Preparation process: into a centrifugal tube added was an aqueous solution of an aptamer (1 equivalent) and a mixed solution (3 equivalents) of a solution of an auristatin tubulin inhibitor in acetonitrile/water in a volume ratio of 1/1; the mixture was kept at a temperature of 37° C. and reacted under stirring for 16 hours.

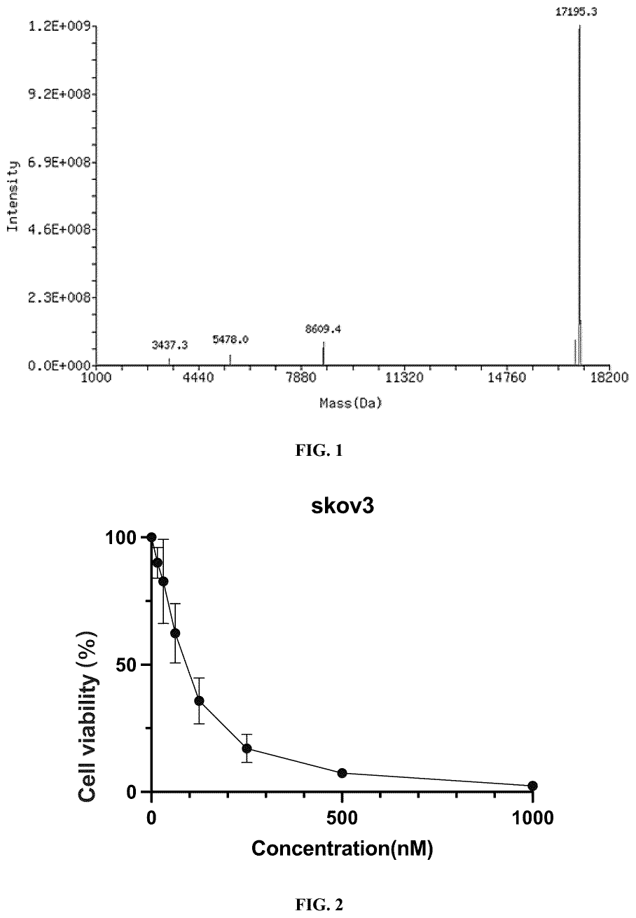

The product was purified by a reversed-phase preparative column and freeze-dried to obtain a C-Met targeted aptamer drug conjugate c-Met-APDC with a yield of about 77%, which was desalted and freeze-dried for later use (the purity be a concentration of 100%). The result of characterization by mass spectrometry is shown in FIG. 1, and MS: Calculated: 17193.0 (Found: 17195.3), indicating the successful preparation of the conjugate as shown above.

### Example 2

The Experiment of c-Met Targeted Aptamer Drug Conjugate (Prepared in Example 1) in Inhibition of Skov3 Cell Proliferation

The specific experimental process was as follows: skov3 cells (purchased from BLUEFBIO) were inoculated into a 96-well plate (1,000 cells per well), cultured at 37° C. under the condition of 5% carbon dioxide for 24 hours, and added with culture media containing different concentrations of a c-Met targeted aptamer drug conjugate c-Met-ApDC. After 24 hours of culture at 37° C. under the condition of 5% carbon dioxide, the drug-containing media were discarded and replaced with drug-free media, and the culture was continued for 96 hours. After a total of 120 hours, the inhibitory effect of the drug was determined by using an MTS kit, and the result is shown in FIG. 2.

As could be seen from FIG. 2, the c-Met targeted aptamer drug conjugate c-Met-ApDC had an excellent inhibitory effect on ovarian cancer skov3 cells, and the IC50 value of the c-Met targeted aptamer drug conjugate c-Met-ApDC was 100 nM. This indicates that highly toxic to tumor cells is at such a low concentration, and the reagent was effective (when the half-maximal inhibitory concentration (IC50) was lower, the drug was more effective, and the toxicity was more significant).

### Example 3

The Experiment of c-Met Targeted Aptamer Drug Conjugate (Prepared in Example 1) in Inhibition of A549 Cell Proliferation

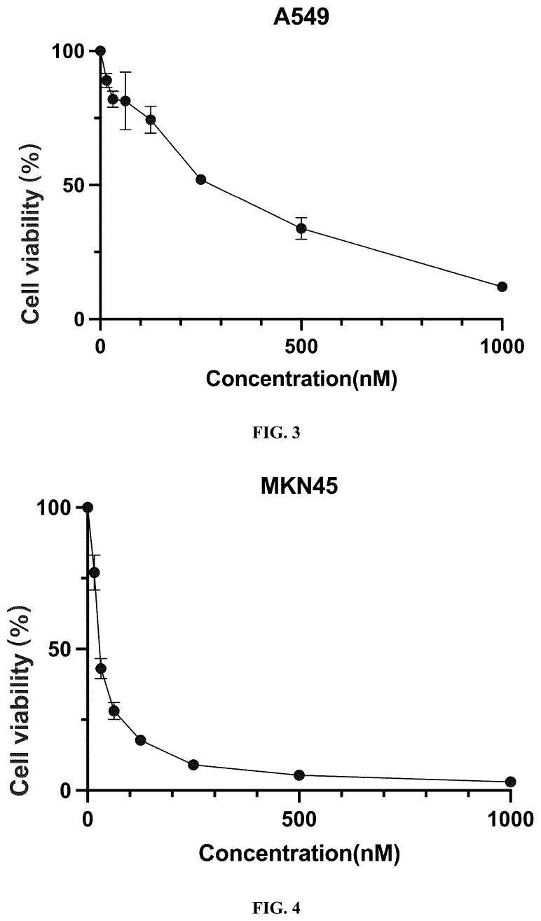

The specific experimental process was as follows: A549 cells were inoculated into a 96-well plate (2,000 cells per well), cultured at 37° C. under the condition of 5% carbon dioxide for 24 hours, and added with culture media containing different concentrations of a c-Met targeted aptamer drug conjugate c-Met-ApDC. After 24 hours of culture at 37° C. under the condition of 5% carbon dioxide, the drug-containing media were discarded and replaced with drug-free media, and the culture was continued for 96 hours. After a total of 120 hours, the inhibitory effect of the drug was determined by using a MTS kit, and the result was as shown in FIG. 3. As could be seen from FIG. 3, the c-Met targeted aptamer drug conjugate c-Met-ApDC had an excellent inhibitory effect on non-small cell lung cancer A549 cells, and the IC50 value of the c-Met targeted aptamer drug conjugate c-Met-ApDC was 250 nM.

### Example 4

The Experiment of c-Met Targeted Aptamer Drug Conjugate (Prepared in Example 1) in Inhibition of MKN45 Cell Proliferation

The specific experimental process was as follows: MKN45 cells were inoculated into a 96-well plate (2,000 cells per well), cultured at 37° C. under the condition of 5% carbon dioxide for 24 hours, and added with culture media containing different concentrations of a c-Met targeted aptamer drug conjugate c-Met-ApDC. After culture at 37° C. under the condition of 5% carbon dioxide for 120 hours, the inhibitory effect of the drug was determined by using a MTS kit. The result was as shown in FIG. 4. As could be seen from FIG. 4, the c-Met targeted aptamer drug conjugate c-Met-ApDC had an excellent inhibitory effect on gastric cancer MKN45 cells, and the IC50 value of the c-Met targeted aptamer drug conjugate c-Met-ApDC was 25 nM.

### Example 5

The Experiment of c-Met Targeted Aptamer Drug Conjugate (Prepared in Example 1) in Inhibition of MUM2b Cell Proliferation

The specific experimental process was as follows: MUM2b cells were inoculated into a 96-well plate (2,000 cells per well), cultured at 37° C. under the condition of 5% carbon dioxide for 24 hours, and added with culture media containing different concentrations of a c-Met targeted aptamer drug conjugate c-Met-ApDC. After 24 hours of culture at 37° C. under the condition of 5% carbon dioxide, the drug-containing media were discarded and replaced with drug-free media, and the culture was continued for 96 hours. After a total of 120 hours, the inhibitory effect of the drug was determined by using an MTS kit. The result was as shown in FIG. 5. As could be seen from FIG. 5, the c-Met targeted aptamer drug conjugate c-Met-ApDC had an excellent inhibitory effect on human highly invasive choroidal melanoma MUM2b cells, and the IC50 value of the c-Met targeted aptamer drug conjugate c-Met-ApDC was 150.5 nM.

### Example 6

Experiment of c-Met Targeted Aptamer Drug Conjugate (Prepared in Example 1) in Triple-Negative Breast Cancer PDX Model of Mice

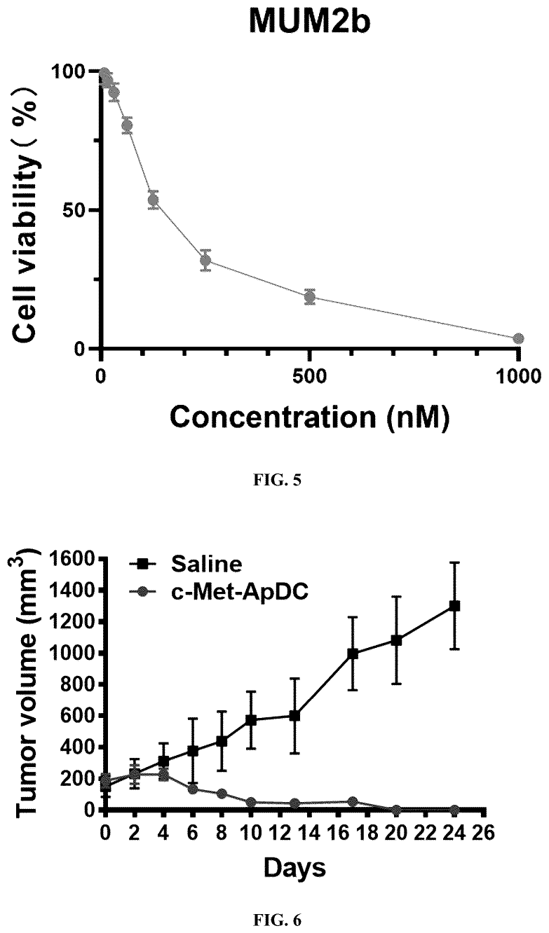

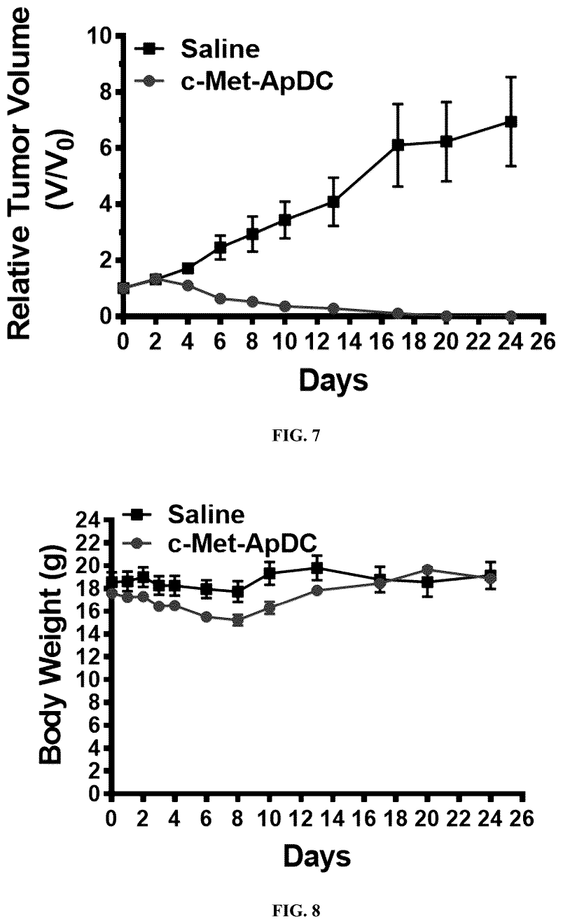

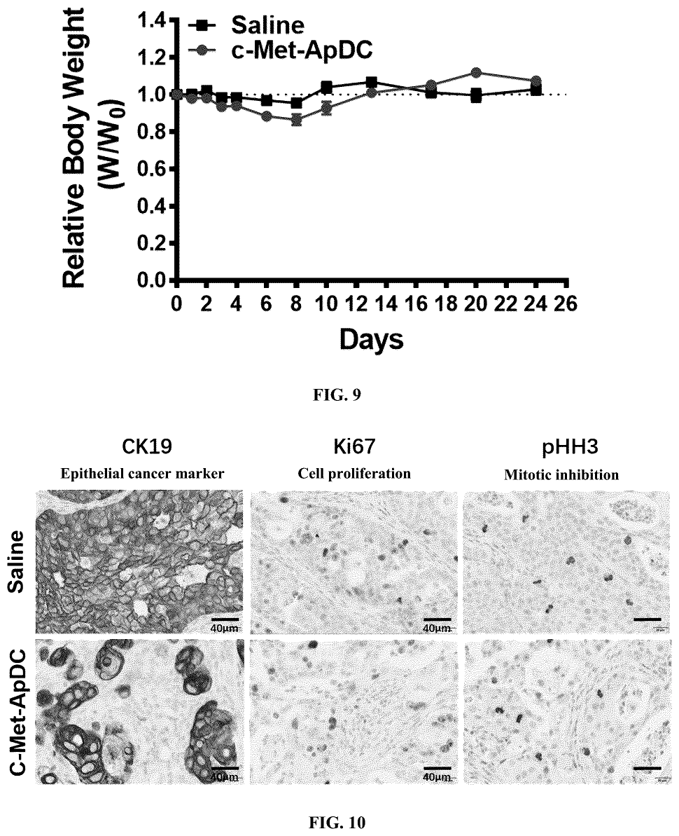

Ten mice with a tumor size of 100-150 mm³ in a triple negative breast cancer PDX model were randomly divided into two groups. In the experiment, the two groups were administrated every other day, wherein the two groups were injected with saline and the c-Met targeted aptamer drug conjugate c-Met-ApDC through the tail vein respectively at a dosage of 0.36 mg/kg equivalent of MMAE (a chemical drug) every other day, for a total of 5 times. The weights of the mice, the lengths of tumors (a), and the widths of tumors (b) were measured and recorded at each injection. The calculation formula of tumor volume (V) was: V=(a×b²)/2. The tumor volume exceeding 1,000 mm³ or the weight loss exceeding 15% was taken as an endpoint of the experiment. The mice were euthanized, and the experiment was terminated. The experimental results were as shown in FIGS. 6 and 7; the tumor inhibition rate of the mice reached 100% (treated for 20 days), and complete healing was achieved. Meanwhile, the weights of the mice were observed. The results are shown in FIGS. 8 and 9, there was no significant difference in the weights of the mice in the group administrated with the c-Met targeted aptamer drug conjugate compared with those in the control group.

### Example 7

Immunohistochemical Characterization of c-Met-ApDC in the Treatment of Tumor in Triple-Negative Breast Cancer PDX Model

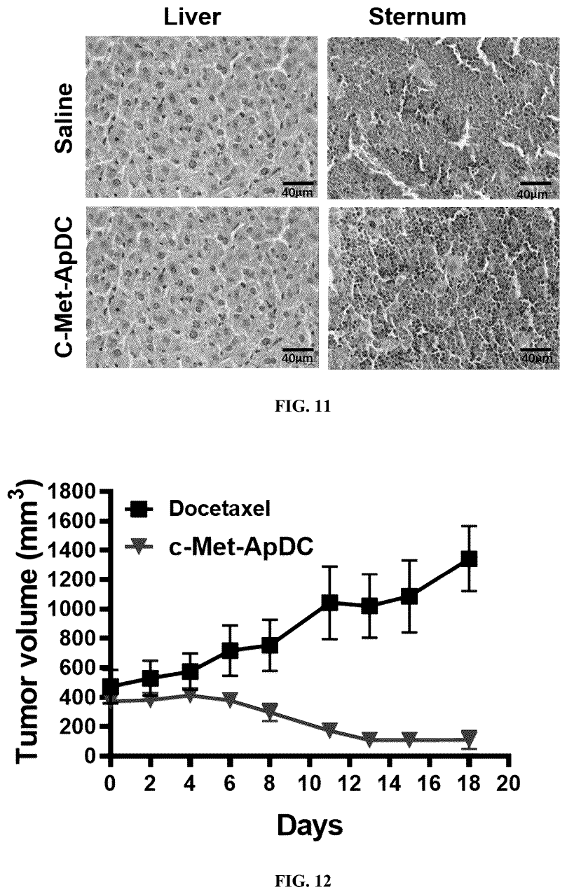

The tumor of Example 5 was embedded and characterized by immunohistochemistry. The result was as shown in FIG. 10. The expression of CK19 (a marker) in tumor tissues was decreased upon treatment with the c-Met targeted aptamer drug conjugate c-Met-ApDC, which indicates that cancer cells were decreased. The expression of a Ki67 marker was decreased and indicated that the proliferation of the tumor cells was effectively inhibited after treatment, and pHH3 was relatively increased, showing that the mitosis of the tumor cells was significantly inhibited after treatment with c-Met-ApDC. The liver and the major organs of the triple-negative breast cancer PDX model after treatment with the c-Met targeted aptamer drug conjugate c-Met-ApDC were embedded and characterized by immunohistochemistry. The results are shown in FIG. 11. After treatment with the c-Met targeted aptamer drug conjugate c-Met-ApDC, there was no apparent inhibition on sternal bone marrow, and there was no significant difference in liver cells compared with the group administrated with saline. It showed that the c-Met targeted aptamer drug conjugate c-Met-ApDC had good safety in the mice in vivo and no apparent liver damage.

The specific embedding process was: into a metal mold put was melted paraffin liquid and a tissue mass; the tissue mass was adjusted with tweezers such that the tissue section was parallel to the bottom of the mold, the mold was covered with a marked cover and filled with the paraffin liquid, and moved to a bench at −10° C. so that the tissue was fixed when the paraffin solidified. After about half an hour, the metal mold was removed to obtain a tissue-containing paraffin block.

### Example 8

Experiment of the c-Met Targeted Aptamer Drug Conjugate Against Large Tumors In Vivo

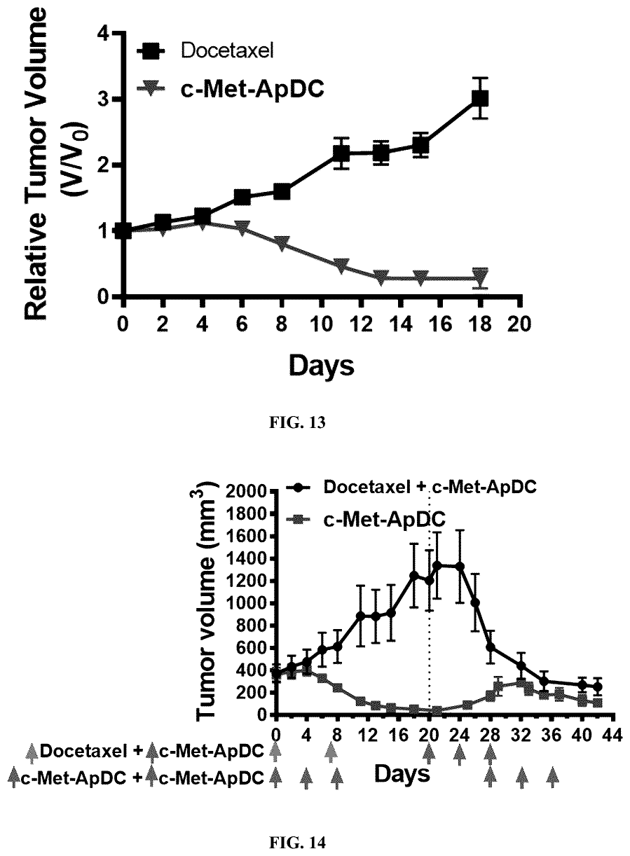

Six mice with a tumor size of 400-500 mm³ in a triple-negative breast cancer PDX model were randomly divided into two groups. In the experiment, the two groups were injected with a clinical first-line chemotherapy drug Docetaxel, and a c-Met targeted aptamer drug conjugate c-Met-ApDC through the tail vein, respectively at a dosage of 20 mg/kg Docetaxel (administrated once every seven days, for two times in total) or 0.36 mg/kg equivalent of MMAE (administrated once every four days, for three times in total). The weights of the mice, the lengths of tumors (a), and the widths of tumors (b) were measured and recorded at each injection. The calculation formula of tumor volume (V) was: V=(a×b²)/2. The tumor volume exceeding 1,500 mm³ or the weight loss exceeding 15% was taken as an endpoint of the experiment. The mice were euthanized, and the experiment was terminated. The experimental results are shown in FIGS. 12 and 13. The large tumors in the mice had been effectively inhibited with an inhibition rate greater than 90.6%. Additionally, as could be seen from FIGS. 12-13, for the chemotherapeutic drug, the volume of the tumor cell still had a rising trend after the two times of administration, and no apparent inhibitory effect could be obtained. On about seven days of administration, the tumor volume in the group administrated with the chemotherapeutic drug began to increase, while the tumor of the group administrated with the reagent of the present invention began to decrease. Over time, the inhibitory rate of the present invention reached greater than 90.6%, while the tumor volume in the group administrated with the chemotherapeutic drug was increased to 3 times the volume, which showed that the present invention had an apparent therapeutic effect compared with the traditional chemotherapeutic drug.

### Example 9

Experiment of the c-Met Targeted Aptamer Drug Conjugate Against Large Tumors In Vivo after Multiple Times of Administration

Six mice with a tumor size of 400-500 mm³ in a triple-negative breast cancer PDX model were randomly divided into two groups. In the experiment, the two groups were injected with a clinical first-line chemotherapy drug, Docetaxel and a c-Met targeted aptamer drug conjugate c-Met-ApDC through the tail vein, respectively, at a dosage of 20 mg/kg Docetaxel (administrated once every seven days, for two times in total) or 0.36 mg/kg equivalent of MMAE (administrated once every four days, for three times in total).

The weights of the mice, the lengths of tumors (a), and the widths of tumors (b) were measured and recorded at each injection. The calculation formula of tumor volume (V) was: V=(a×b²)/2. The tumor volume exceeding 1,500 mm³ or the weight loss exceeding 15% was taken as an endpoint of the experiment. The mice were euthanized, and the experiment was terminated. In the group administrated with Docetaxel, 0.36 mg/kg equivalent of MMAE was injected into the mice through the tail vein once every four days continuously for three times after the tumor volume exceeded 1,500 mm³. As shown in FIG. 14, the tumor volume of the mice in the group administrated with Docetaxel alone was gradually increased. Still, the mice were injected continuously with the drug conjugate of the present invention after the tumor volume exceeded 1,500 mm³ so that the tumor volume was significantly reduced. It was almost less than 200 mm³ after 44 days. In the group administrated with c-Met-APDC, the tumor recurrence of the mice was also effectively inhibited; the tumor was almost eliminated in 20 days, and the tumor was inhibited again when it recurred in the later stage and wholly eliminated after 50 days.

### Example 10

Experiment of Mice with Different Administration Dosages and Frequencies of c-Met-ApDC

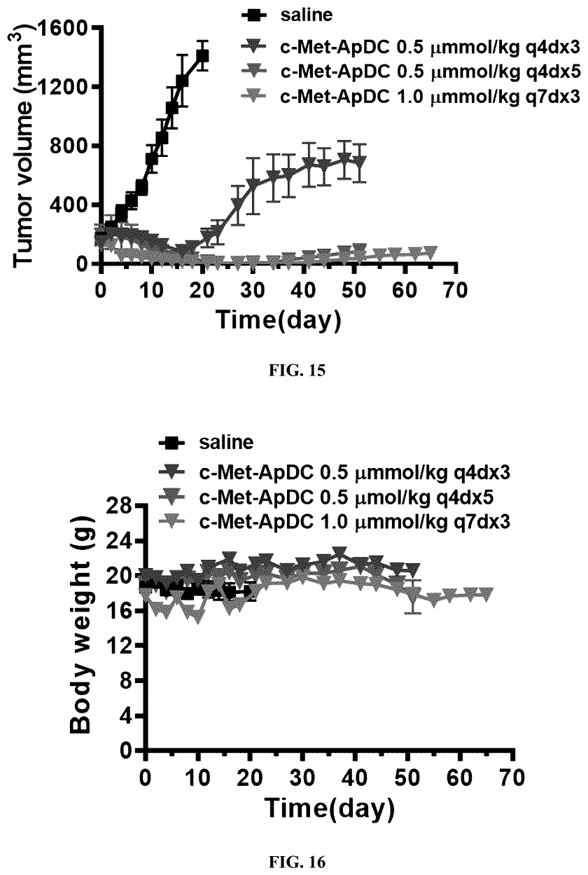

Twenty mice with a tumor size of 100-200 mm³ in a triple-negative breast cancer PDX model were randomly divided into four groups. In the experiment, the four groups were injected with saline; the C-Met targeted aptamer drug conjugate c-Met-ApDC at a dosage of 0.5 μmol/kg (administrated once every four days, for a total of 3 times); the C-Met targeted aptamer drug conjugate c-Met-ApDC at a dosage of 0.5 μmol/kg (administrated once every four days, for a total of 5 times); and the C-Met targeted aptamer drug conjugate c-Met-ApDC at a dosage of 1.0 μmol/kg (administrated once every seven days, for a total of 3 times) through tail vein respectively. The weights of the mice, the lengths of tumors (a), and the widths of tumors (b) were measured and recorded every other day. The calculation formula of tumor volume (V) was: V=(a×b²)/2. The tumor volume exceeding 1,500 mm³ or the weight loss exceeding 15% was taken as an endpoint of the experiment. The mice were euthanized, and the experiment was terminated. The experimental results were as shown in FIG. 15. Different dosages of c-Met-ApDC all showed good tumor inhibitory activity. At the dosage of 0.5 μmol/kg (administrated once every four days, for a total of 3 times), a tumor inhibitory effect more significant than 70% was achieved, and the tumor inhibitory effect of 1.0 μmol/kg of c-Met-ApDC was evident on the mice, with a small amount of recurrence seen after 65 days of observation. The weights of the mice in the group administrated with ApDC were stable, indicating that multiple dosages of c-Met-ApDC had good safety (as shown in FIG. 16).

### Example 11

The Experiment of c-Met Targeted Aptamer Drug Conjugate Against HT-29 Tumor In Vivo

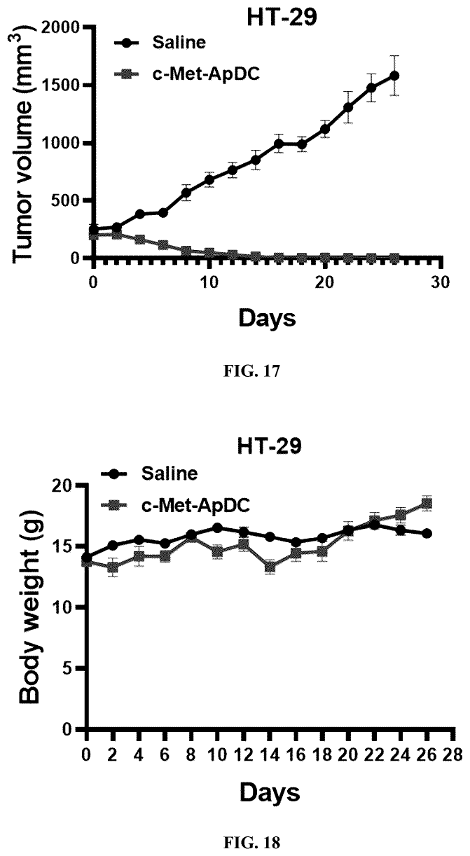

Ten mice with a tumor size of 200-300 mm³ in a human colon cancer cell HT-29-bearing mouse model were randomly divided into two groups. In the experiment, the two groups were injected with saline and the c-Met targeted aptamer drug conjugate c-Met-ApDC through the tail vein respectively at a dosage of 0.5 μmol/kg (administrated once every four days, for a total of 5 times). The weights of the mice, the lengths of tumors (a), and the widths of tumors (b) were measured and recorded every other day. The calculation formula of tumor volume (V) was: V=(a×b²)/2. The tumor volume exceeding 1,500 mm³ or the weight loss exceeding 15% was taken as an endpoint of the experiment. The mice were euthanized, and the experiment was terminated. The experimental results are shown in FIG. 17. c-Met-ApDC could effectively inhibit HT-29 tumor proliferation, the tumor inhibition rate of the mice reached 100% (on day 10), and complete healing was achieved. Meanwhile, the weights of the mice were observed. The results were shown in FIG. 18. There was no significant difference in the weights of the mice in the group administrated with the c-Met targeted aptamer drug conjugate compared with those in the control group.

### Example 12

The Experiment of c-Met Targeted Aptamer Drug Conjugate Against A549 Tumor In Vivo

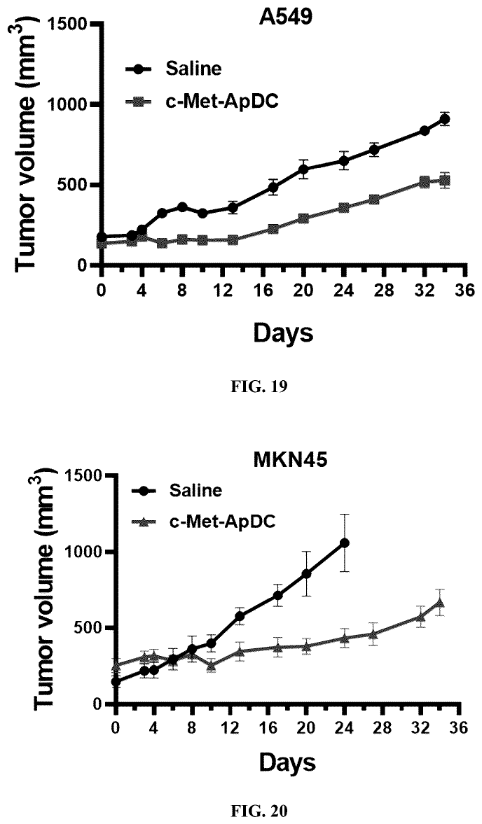

Eight mice with a tumor size of 100-200 mm³ in a human lung cancer A549 cell-bearing mouse model were randomly divided into two groups. In the experiment, the two groups were injected with saline and the c-Met targeted aptamer drug conjugate c-Met-ApDC through the tail vein respectively at a dosage of 0.5 μmol/kg (administrated once every four days, for a total of 3 times). The weights of the mice, the lengths of tumors (a), and the widths of tumors (b) were measured and recorded every other day. The calculation formula of tumor volume (V) was: V=(a×b²)/2. The tumor volume exceeding 1,500 mm³ or the weight loss exceeding 15% was taken as an endpoint of the experiment. The mice were euthanized, and the experiment was terminated. The experimental results are shown in FIG. 19. c-Met-ApDC could effectively inhibit A549 tumor proliferation, the tumor inhibition rate of the mice reached 42.3%, and the tumor inhibition rate TGI=(1−tumor volume in the treatment group/tumor volume in the control group)*100%.

### Example 13

The Experiment of c-Met Targeted Aptamer Drug Conjugate Against MKN45 Tumor In Vivo

Eight mice with a tumor size of 150-250 mm³ in a human gastric cancer cell MKN45-bearing mouse model were randomly divided into two groups. In the experiment, the two groups were injected with saline and the c-Met targeted aptamer drug conjugate c-Met-ApDC through the tail vein respectively at a dosage of 0.5 μmol/kg (administrated once every four days, for a total of 3 times). The weights of the mice, the lengths of tumors (a), and the widths of tumors (b) were measured and recorded every other day. The calculation formula of tumor volume (V) was: V=(a×b²)/2. The tumor volume exceeding 1,500 mm³ or the weight loss exceeding 15% was taken as an endpoint of the experiment. The mice were euthanized, and the experiment was terminated. The experimental results are shown in FIG. 20. The c-Met-ApDC could effectively inhibit MKN45 tumor proliferation, and the tumor inhibition rate of the mice reached 59.2%.

### Example 14

The Experiment of c-Met Targeted Aptamer Drug Conjugate Against Choroidal Melanoma Tumor MUM2b In Vivo

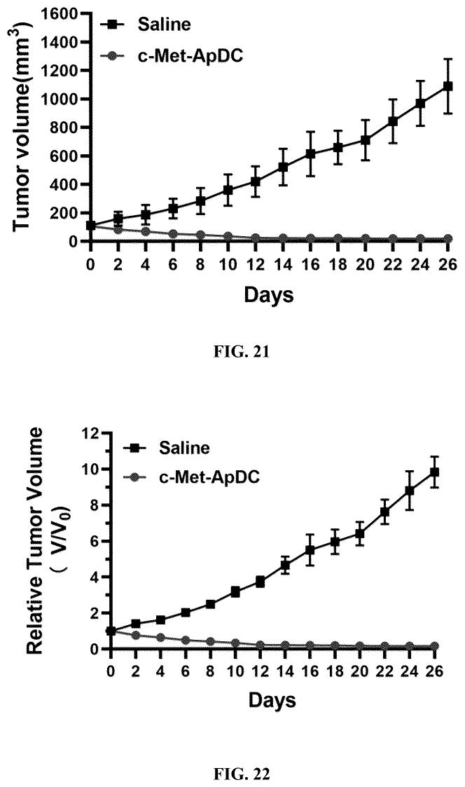

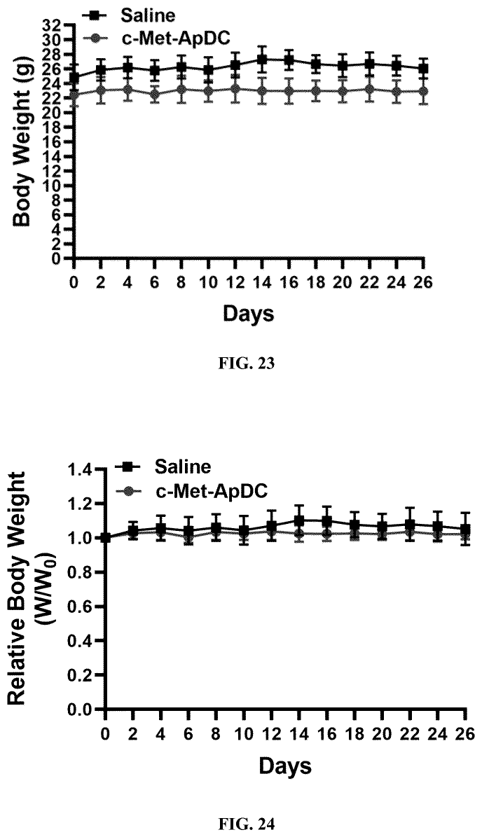

Eight mice with a tumor size of 100-150 mm³ in a human choroidal melanoma MUN2b-bearing mouse model were randomly divided into two groups. In the experiment, the two groups were injected with saline and the c-Met targeted aptamer drug conjugate c-Met-ApDC through the tail vein respectively at a dosage of 0.5 μmol/kg (administrated once every four days, for a total of 4 times). The weights of the mice, the lengths of tumors (a), and the widths of tumors (b) were measured and recorded every other day. The calculation formula of tumor volume (V) was: V=(a×b²)/2. The tumor volume exceeding 1,500 mm³ or the weight loss exceeding 15% was taken as an endpoint of the experiment. The mice were euthanized, and the experiment was terminated. The experimental results are shown in FIGS. 21 and 22. c-Met-ApDC could effectively inhibit MUM2b tumor proliferation, the tumor inhibition rate of the mice reached 98.13%, and the tumor inhibition rate TGI=(1−tumor volume in the treatment group/tumor volume in the control group)*100%. Meanwhile, the weights of the mice were observed. The results are shown in FIGS. 23 and 24, there was no significant difference in the weights of the mice in the group administrated with the c-Met targeted aptamer drug conjugate compared with those in the control group.

### Example 15

The Experiment of c-Met Targeted Aptamer Drug Conjugate Against In Situ Choroidal Melanoma Tumor MUM2b In Vivo

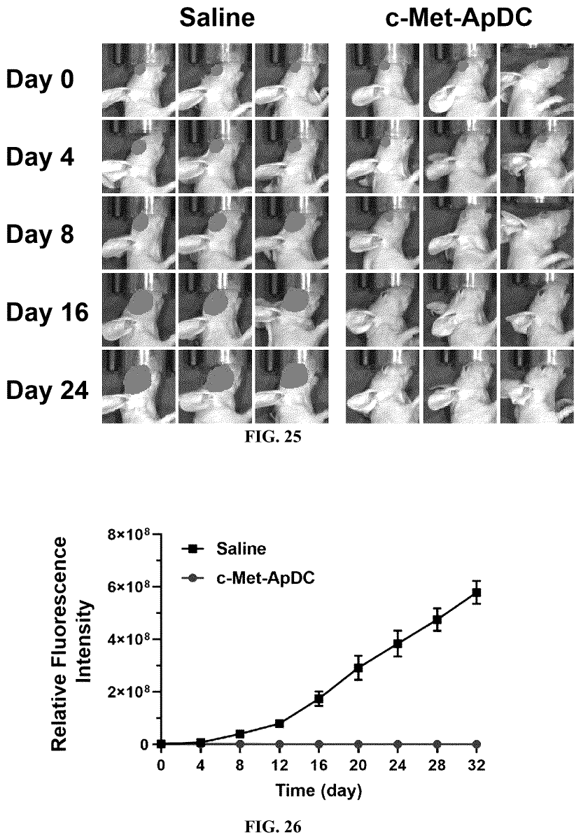

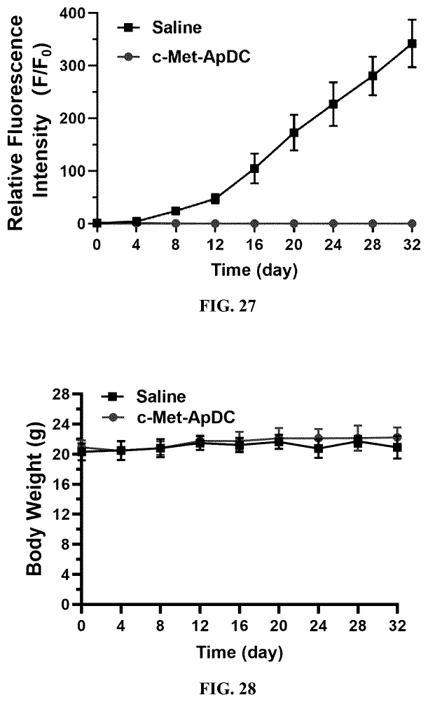

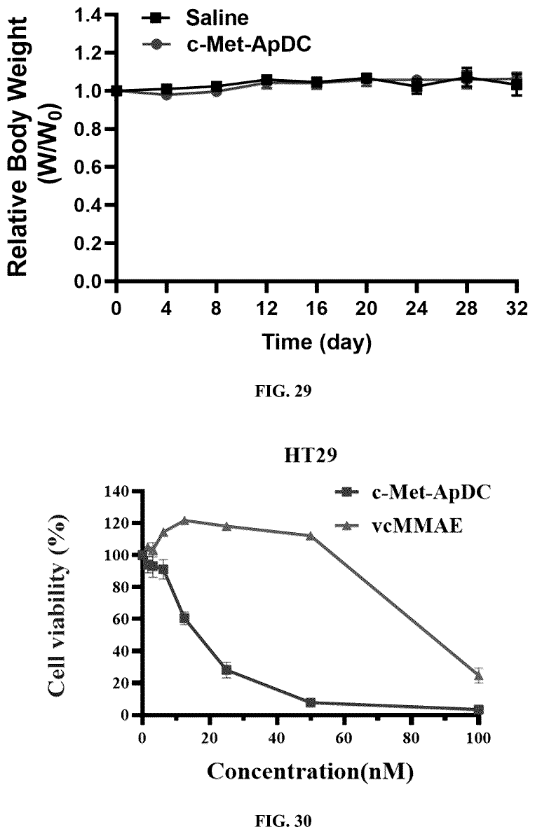

2*10\hat{ }5 human choroidal melanoma cells MUM2b expressing Luciferase were injected into the subretinal cavity. Twelve mice with 1-10\hat{ }5 fluorescent signals of ocular tumors were selected one week later and randomly divided into two groups. In the experiment, the two groups were intravitreally injected with saline and the c-Met targeted aptamer drug conjugate c-Met-ApDC at a dosage of 500 μM/2 μL (once in total). Every four days, the mice were injected with fluorescein sodium D intraperitoneally, and the tumor fluorescence signals in the eyes of the mice and the weight of the mice were recorded by an in vivo imager. The tumor eyes losing eyeball morphology or weight loss greater than 15% was taken as the endpoint of the experiment. The mice were euthanized, and the experiment was terminated. As shown in FIGS. 25, 26, and 27, c-Met-ApDC could inhibit 100% of the proliferation of ocular in situ MUM2b tumors. Meanwhile, the weights of the mice were observed. The results are shown in FIGS. 28 and 29, there was no significant difference in the weights of the mice in the group administrated with the c-Met targeted aptamer drug conjugate compared with those in the control group.

### Comparative Example 1

Experiment for Comparing c-Met Targeted Aptamer Drug Conjugate and vcMMAE in Inhibition of HT29 Cell Proliferation

The specific experimental process was as follows: HT29 cells were inoculated into a 96-well plate (2,000 cells per well), cultured at 37° C. under the condition of 5% carbon dioxide for 24 hours, and added with culture media containing different concentrations of a c-Met targeted aptamer drug conjugate c-Met-ApDC and the same concentration of the drug vcMMAE. After culture at 37° C. under the condition of 5% carbon dioxide for 120 hours, the inhibitory effect of the drug was determined by using a MTS kit, and the result was as shown in FIG. 30. It could be seen from FIG. 30 that both the c-Met targeted aptamer drug conjugate c-Met-ApDC and the drug vcMMAE had good inhibitory effects on the HT29 cells, but conjugating with the c-Met aptamer significantly improved the anti-tumor activity of the drug vcMMAE. As could be seen from FIG. 30, almost 100% proliferation inhibition of the HT29 cells was achieved at the dosage of 40 nM. However, vcMMAE alone could not effectively inhibit as such; such an improvement in activity might be the effect of the combined action of the aptamer and the drug. The combined use of drugs could continuously reduce cell proliferation. In contrast, the drug vcMMAE alone achieved enhanced proliferation first and then weakened proliferation, indicating that the aptamer of the present invention has a unilateral effect of improving the pharmaceutical effect.

### Comparative Example 2

Experiment for Comparing c-Met Targeted Aptamer Drug Conjugate and vcMMAE in Inhibition of MKN45 Cell Proliferation

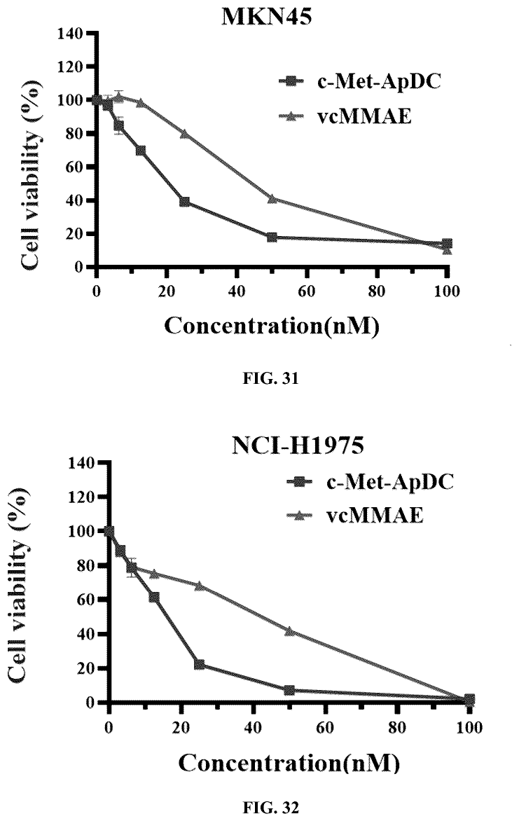

The specific experimental process was as follows: MKN45 cells were inoculated into a 96-well plate (2,000 cells per well), cultured at 37° C. under the condition of 5% carbon dioxide for 24 hours, and added with culture media containing different concentrations of a c-Met targeted aptamer drug conjugate c-Met-ApDC and the same concentration of the drug vcMMAE. After culture at 37° C. under the condition of 5% carbon dioxide for 120 hours, the inhibitory effect of the drug was determined by using a MTS kit, and the result was as shown in FIG. 31. As could be seen in FIG. 31, the c-Met targeted aptamer drug conjugate c-Met-ApDC and the drug vcMMAE had good inhibitory effects on MKN45 cells, and conjugating with the c-Met aptamer significantly improved the anti-tumor activity of the drug vcMMAE, which might be due to that the firm binding of the aptamer and the target enhances the pharmaceutical effect, or might be a result of a combined action.

### Comparative Example 3

Experiment for Comparing c-Met Targeted Aptamer Drug Conjugate and vcMMAE in Inhibition of NCI-H1975 Cell Proliferation

The specific experimental process was as follows: NCI-H1975 cells were inoculated into a 96-well plate (2,000 cells per well), cultured at 37° C. under the condition of 5% carbon dioxide for 24 hours, and added with culture media containing different concentrations of a c-Met targeted aptamer drug conjugate c-Met-ApDC and the same concentration of the drug vcMMAE. After culture at 37° C. under the condition of 5% carbon dioxide for 120 hours, the inhibitory effect of the drug was determined by using an MTS kit, and the result is shown in FIG. 32. As could be seen from FIG. 32, the c-Met targeted aptamer drug conjugate c-Met-ApDC and the drug vcMMAE had good inhibitory effects on NCI-H1975 cells, and conjugating with the c-Met aptamer significantly improved the anti-tumor activity of the drug vcMMAE.

### Comparative Example 4

Experiment for Comparing c-Met Targeted Aptamer Drug Conjugate and vcMMAE in Inhibition of MUM2b Cell Proliferation

The specific experimental process was as follows: MUM2b cells were inoculated into a 96-well plate (2,000 cells per well), cultured at 37° C. under the condition of 5% carbon dioxide for 24 hours, and added with culture media containing different concentrations of a c-Met targeted aptamer drug conjugate c-Met-ApDC and the same concentration of the drug vcMMAE. After culture at 37° C. under the condition of 5% carbon dioxide for 120 hours, the inhibitory effect of the drug was determined by using an MTS kit, and the result is shown in FIG. 33. As could be seen from FIG. 33, the c-Met targeted aptamer drug conjugate c-Met-ApDC and the drug vcMMAE had good inhibitory effects on MUM2b cells, and conjugating with the c-Met aptamer significantly improved the anti-tumor activity of the drug vcMMAE.

### Comparative Example 5

### Experiment for Comparing Stability of Drug Conjugated at 3-Terminal and Drug Conjugated at 5-Terminal

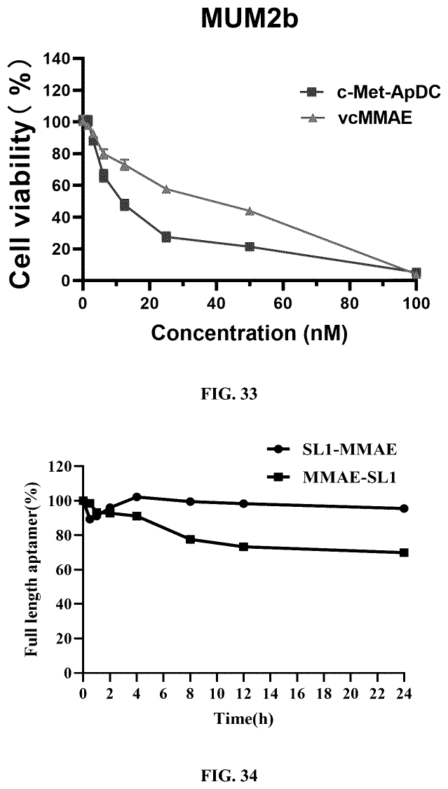

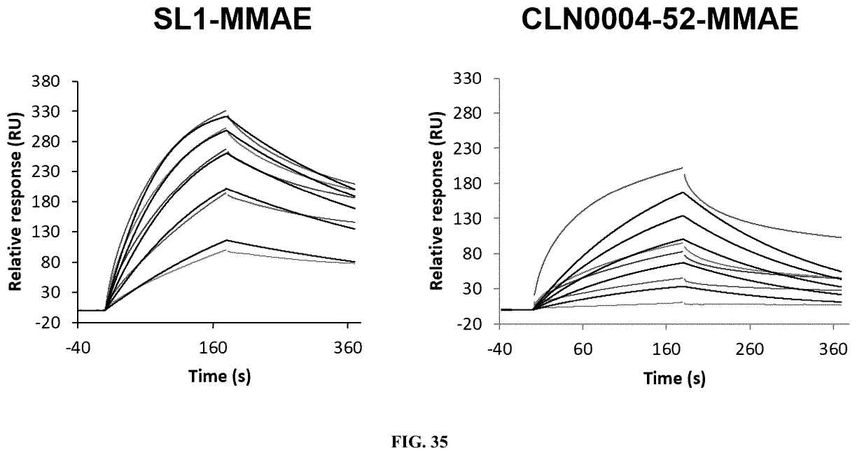

The specific experimental process was as follows: a drug conjugated at the 3-terminal MMAE-SL1 and a drug conjugated at the 5-terminal SL1-MMAE were incubated in 10% FBS at 37° C. to determine the stability of the two drugs (the specific synthesis method was the same as that in Example 1, except that the position of nucleic acid modification was different). Samples were taken at 0, 0.5, 1, 2, 4, 8, 12, and 24 h, respectively, and the stability of the two drugs was characterized by gel electrophoresis. The results are shown in FIG. 34, the stability of the drug conjugated at the 3-terminal SL1-MMAE was significantly improved compared with that of the drug conjugated at the 5-terminal. At 24 hours, the full-length sequence of the 3-terminal nucleic acid remained at about 100%. There was almost no cleavage, while the full-length sequence of the 5-terminal remained at only a little more than 60%, which showed that the cleavage rate of the drug conjugate with 5-terminal modification was about 30.1%-40% under the same conditions. For the drug conjugated at the 5-terminal, the nucleic acid was almost wholly degraded or cracked after more than 48 hours, while for the drug conjugated at the 3-terminal, the nucleic acid remained in a stable state without cracking after 48 hours, and the degradation rate was meager of only about 4.4%. This showed that the stability of SL1-MMAE modified with the drug at the 3-terminal was significantly higher than that of MMAE-SL1 modified with the drug at the 5-terminal. Therefore, the c-Met ApDC modified with MMAE at the 3-terminal was a more stable preferred drug, which could effectively prevent drug degradation during transportation and prolong the degradation half-life in vivo. Additionally, when the aptamer kept a complete full-length sequence longer, then the half-life in vivo of the aptamer would be longer. The aptamer could be transported to a designated target, the effective drug duration would be longer, and the effect would last longer so that the full-length sequence could bind to the target protein effectively. On the contrary, for the drug conjugate with the 5-terminal modification, the nucleic acid was degraded by 40% within 24 hours, it was planned to be entirely degraded by 48 hours, only a tiny amount of the drug conjugate would exist, and most of the drug was the drug MMAE alone, so that the pharmaceutical effect was naturally decreased (almost no nucleic acid bound to the target protein, and naturally the effect was significantly reduced), the half-life in vivo was very short, and some of the drug conjugate might be cracked without being transported to the target.

### Comparative Example 6

Experiment for Comparing the Binding Affinity of Different c-Met Aptamer Drug Conjugates with c-Met Protein

The specific experimental process was as follows: the c-Met protein-bound aptamer CLN0004-52 (sequence SEQ: ID2: GAGTGCGTAATGGTACGATTTGGGAAGTGGCTTGGGGTGGGATTAGTTTTGG) reported in the literature was selected and modified with a mercapto group at the 3-terminal and then conjugated with vcMMAE to construct the c-Met targeted aptamer drug conjugate CLN0004-52-MMAE. The affinity of CLN0004-52-MMAE and SL1-MMAE with a c-Met protein was characterized by surface plasmon resonance spectroscopy. The binding forces of two c-Met ApDCs were determined at concentrations of 100, 200, 300, 400, and 500 nM. The results are shown in FIG. 35, the kD value of SL1-MMAE was 77.5 nM, which was much lower than that of CLN0004-52-MMAE (21.5 μm), indicating that SL1-MMAE had a higher binding force than other c-Met targeted ApDCs, and it was a preferred c-Met ApDC. Strong affinity stated that it could bind with c-Met more strongly, enhancing the therapeutic effect.

### Comparative Example 7

Experiment for Comparing Cytotoxicity of Different c-Met Aptamer Drug Conjugates

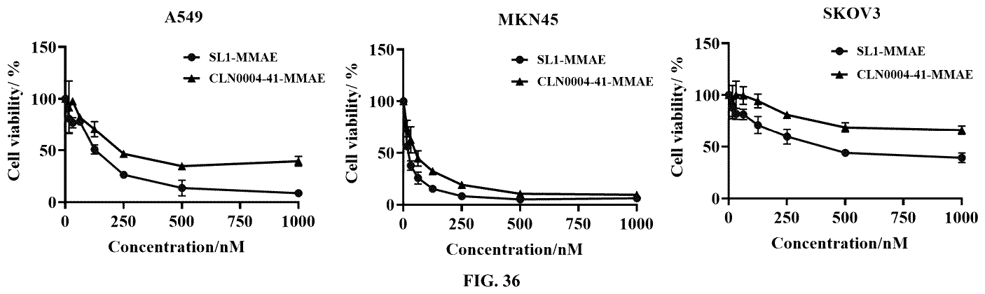

The specific experimental process was as follows: the c-Met protein-bound aptamer CLN0004-41 (sequence SEQ: ID2: GAGTGCGTAATGGTACGATTTGGGAAGTGGCTTGGGGTGGG) reported in the literature was selected, and modified with a mercapto group at the 3-terminal and then conjugated with vcMMAE to construct the c-Met targeted aptamer drug conjugate CLN0004-41-MMAE. The cytotoxicity of CLN0004-41-MMAE and SL1-MMAE in cell lines with high expression of c-Met was characterized by a CCK-8 method. The cytotoxicity of two c-Met ApDCs at concentrations of 15.625 nM, 31.25 nM, 62.5 nM, 125 nM, 250 nM, 500 nM, and 1,000 nM was determined. The result is shown in FIG. 36, and the IC50 values of SL1-MMAE were (A549: 119.5 nM, MKN45: 19.36 nM, SKOV3: 442.9 nM), which were all significantly lower than the IC50s of CLN0004-41-MMAE (A549: 327 nM, MKN45: 51.91 nM, SKOV3: 1771 nM), indicating that SL1-MMAE had higher cytotoxicity than other c-Met targeted ApDC, and it was a preferred c-Met ApDC.

To sum up, the c-Met targeted aptamer drug conjugate in the present invention has high efficiency in vivo tumor inhibition activity, and its inhibition rate on some tumors is as high as 100% to achieve complete healing. Meanwhile, the inhibition experiment against large tumors in vivo shows that compared with the standard chemotherapy Docetaxel group, the drug conjugate has a pronounced inhibition effect and achieves high-efficiency inhibition of 90.6%. The tumor inhibition rate on the tumors in the human colon cancer cell HT-29-bearing mouse model is as high as 100%, which achieves complete healing; the tumor inhibition rate on the tumors in the human lung cancer A549 cell-bearing mouse model reached 42.3%, which effectively inhibits tumor proliferation; the tumor inhibition rate on the tumors in the human gastric cancer cell MKN45-bearing mouse model reached 59.2%, which effectively inhibits tumor proliferation; the tumor inhibition rate on the tumors in the human choroidal melanoma MUN2b-bearing mouse model advanced 98.13%; and 100% tumor inhibition is achieved by intravitreal administration to the human choroidal melanoma MUN2b-bearing mouse model. This also fully shows that adopting the c-Met targeted aptamer drug conjugate of the present invention has different inhibitory effects when administrated to different tumors and in various modes of administration. For example, it has an inhibitory effect of 100% on the tumor of triple-negative breast cancer and can achieve the purpose of treatment. It also has the effect of significantly reducing the tumor volume for a large tumor volume.

The examples above merely illustrate the principles and efficacy of the present invention rather than limiting the present application. Anyone skilled in the art can modify or change the examples above without departing from the spirit and scope of the present application. Therefore, all equivalent modifications or changes made by those of ordinary skills in the art without departing from the spirit and technical idea disclosed in the present invention should still be covered by the claims of the present application.

1. A method for treating a tumor, comprising applying a drug conjugate to a subject so that the conjugate binds to a c-Met protein in the body of the subject in a targeted manner, thereby bringing the drug conjugate into a tumor cell to inhibit the proliferation of the tumor cell, wherein the subject comprises human and a mammal; and the drug conjugate comprises an aptamer SL1 and a tubulin inhibitor.

2. According to claim 1, the method wherein a sequence of the nucleic acid aptamer SL1 is shown in SEQ ID NO:1.

3. The method, according to claim 2, wherein the tubulin inhibitor is selected from an auristatin tubulin inhibitor.

4. The method, according to claim 3, wherein the conjugating is selected from covalent conjugating.

5. The method according to claim 4, wherein the tubulin inhibitor further comprises a first linking group for linking the aptamer, and the aptamer further includes a second linking group for linking the tubulin inhibitor; the first linking group is selected from mercapto or amino, and the second linking group is assigned from carboxyl or azido, so that the first group is linked with the second group, thereby covalently conjugating the aptamer SL1 and the tubulin inhibitor.

6. according to claim 5, the method wherein the aptamer is conjugated with tubulin inhibitor after stability modification; the stability modification is a 3-terminal modification, and the terminal modification is selected from mercapto modification or amino modification.

7. The method according to claim 6, wherein a chemical formula of the drug conjugate is selected from

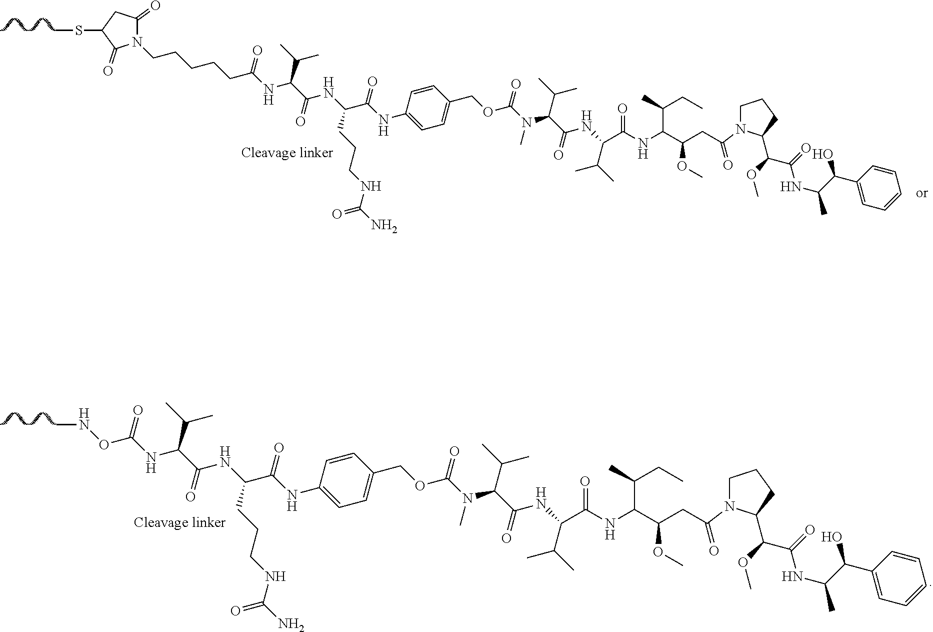 [SMILES: CC[C@H](C)C(NC(=O)[C@@H](NC(=O)[C@H](C(C)C)N(C)C(=O)OCc1ccc(NC(=O)[C@H](CCCNC(N)=O)NC(=O)[C@@H](NC(=O)CCCCCN2C(=O)CC(SC)C2=O)C(C)C)cc1)C(C)C)[C@@H](CC(=O)N1CCC[C@H]1[C@H](OC)C(=O)N[C@H](C)[C@@H](O)c1ccccc1)OC ; CC[C@H](C)C(NC(=O)[C@@H](NC(=O)[C@H](C(C)C)N(C)C(=O)OCc1ccc(NC(=O)[C@H](CCCNC(N)=O)NC(=O)[C@@H](NC(=O)ONC)C(C)C)cc1)C(C)C)[C@@H](CC(=O)N1CCC[C@H]1[C@H](OC)C(=O)N[C@H](C)[C@@H](O)c1ccccc1)OC]

8. The method, according to claim 7, wherein the tumor is selected from a malignant tumor with high expression of c-Met.

9. The method, according to claim 8, wherein the tumor with high expression of c-Met is selected from triple-negative breast cancer, colon cancer, lung cancer, gastric cancer, or choroidal melanoma.

10. The method, according to claim 9, wherein the tumor with high expression of c-Met is triple-negative breast cancer, colon cancer, or choroidal melanoma.

11. The method, according to claim 10, when the choroidal melanoma is treated, an administration mode is intravenous administration or intravitreal administration.

12. A drug conjugate for treating a tumor, comprising the aptamer SL1 and the tubulin inhibitor that are conjugated.

13. According to claim 12, the drug conjugate wherein a sequence of the aptamer SL1 is shown in SEQ ID NO:1.

14. The drug conjugate, according to claim 13, wherein the tubulin inhibitor is selected from an auristatin tubulin inhibitor.

15. according to claim 14, the drug conjugate wherein the auristatin tubulin inhibitor is selected from one of monomethyl auristatin E, monomethyl auristatin F, auristatin-0101, or derivatives thereof.

16. The drug conjugate, according to claim 15, wherein the conjugating is selected from covalent conjugating.

17. The drug conjugate, according to claim 16, wherein the tubulin inhibitor further comprises a first linking group for linking the aptamer, the aptamer further includes a second linking group for linking the tubulin inhibitor. The first linking group is selected from mercapto or amino, and the second linking group is assigned from carboxyl or azido.

18. according to claim 17, the drug conjugate wherein the aptamer is conjugated with the tubulin inhibitor after stability modification; the stability modification is terminal, and the terminal modification is selected from mercapto modification or amino modification.

19. The drug conjugate, according to claim 18, wherein the aptamer is modified to have a modifying group at a 3′ end.

20. The drug conjugate, according to claim 19, wherein a chemical formula of the drug conjugate is selected from

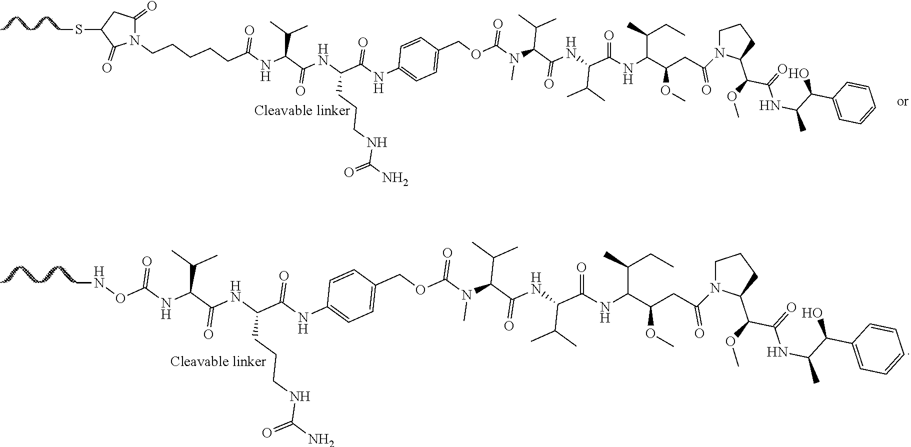 [SMILES: CC[C@H](C)C(NC(=O)[C@@H](NC(=O)[C@H](C(C)C)N(C)C(=O)OCc1ccc(NC(=O)[C@H](CCCNC(N)=O)NC(=O)[C@@H](NC(=O)CCCCCN2C(=O)CC(SC)C2=O)C(C)C)cc1)C(C)C)[C@@H](CC(=O)N1CCC[C@H]1[C@H](OC)C(=O)N[C@H](C)[C@@H](O)c1ccccc1)OC ; CC[C@H](C)C(NC(=O)[C@@H](NC(=O)[C@H](C(C)C)N(C)C(=O)OCc1ccc(NC(=O)[C@H](CCCNC(N)=O)NC(=O)[C@@H](NC(=O)ONC)C(C)C)cc1)C(C)C)[C@@H](CC(=O)N1CCC[C@H]1[C@H](OC)C(=O)N[C@H](C)[C@@H](O)c1ccccc1)OC]

21. The drug conjugate, according to claim 20, wherein the tumor is selected from one or more triple-negative breast cancer, colon cancer, lung cancer, gastric cancer, or choroidal melanoma.


SEQUENCE LISTING (2 sequences)

SEQ ID NO: 1 - DNA, 50 bp, synthetic construct
  source 1..50: mol_type = other DNA
atcaggctggatggtagctcggtcggggtgggtgggttggcaagtctgat

SEQ ID NO: 2 - DNA, 52 bp, synthetic construct
  source 1..52: mol_type = other DNA
gagtgcgtaatggtacgatttgggaagtggcttggggtgggattagttttgg